(19) United States
(12) Patent Application Publication
(10) Pub. No.: US 2023/0363257 A1
(43) Pub. Date: Nov. 9, 2023

(54) ORGANIC MIXTURES AND APPLICATIONS THEREOF IN ORGANIC ELECTRONIC DEVICES

(71) Applicant: Zhejiang Brilliant Optoelectronic Technology Co.,Ltd., Taizhou (CN)

(72) Inventors: Junyou PAN, Taizhou (CN); Lei WEN, Taizhou (CN); Caifa PAN, Taizhou (CN)

(73) Assignee: Zhejiang Brilliant Optoelectronic Technology Co.,Ltd., Taizhou (CN)

(21) Appl. No.: 18/346,454

(22) Filed: Jul. 3, 2023

Related U.S. Application Data
(63) Continuation of application No. PCT/CN2022/070045, filed on Jan. 4, 2022.

(30) Foreign Application Priority Data
Jan. 4, 2021 (CN) 202110002334.2

(51) Int. Cl. H10K 85/60

(52) U.S. Cl. CPC H10K 85/615; H10K 85/6574; H10K 2101/30

(57) ABSTRACT

Organic mixtures including a first organic compound H₁ and a second organic compound H₂ are disclosed. Also provided are formulations including at least one organic mixture and at least one organic solvent. Further provided are organic electronic devices including at least one organic mixture.

### CROSS-REFERENCE TO RELATED APPLICATIONS

The present application is a continuation of International Application No. PCT/CN2022/070045, filed on Jan. 4, 2022, which claims priority to Chinese Patent Application No. 202110002334.2, filed on Jan. 4, 2021. All of the aforementioned applications are incorporated herein by reference in their entireties.

### TECHNICAL FIELD

The present disclosure relates to the field of organic electroluminescent technology, and in particularly to an organic mixture, and the applications thereof in the organic electronic field, particularly in the electroluminescent field.

### BACKGROUND

Due to the diversity of synthesis, low manufacturing costs, excellent optical, and electrical properties, organic light-emitting diodes (OLEDs) have great potential for the realization of novel optoelectronic devices, such as in flat-panel displays and lighting applications.

The organic electroluminescent phenomenon refers to a phenomenon of converting electrical energy to photonic energy with organic substance. An organic electroluminescent element utilizing the organic electroluminescent phenomenon usually has a structure comprising an anode, a cathode, and an organic layer therebetween. In order to improve the efficiency and lifetime of the organic electroluminescent element, the organic layer has a multi-layer structure, and each layer comprises different organic substances. For example, each layer can be a hole-injection layer, a hole-transport layer, a light-emitting layer, an electron-transport layer, an electron-injection layer, etc. When a voltage is applied between two electrodes of such an organic electroluminescent element, holes are injected into the organic layer from the anode, electrons are injected into an organic layer from the cathode; and an exciton is formed when an injected hole and an injected electron recombine in the emission layer. The exciton emits light when it transitions back to the ground state. The organic electroluminescent element has characteristics of self-emission, high luminance, high efficiency, low driving voltage, wide viewing angle, high contrast, high responsivity, etc.

In order to improve the luminescence efficiency of organic electroluminescent elements, various emitting material systems of fluorescence and phosphorescence have been developed; and the development of the excellent blue emissive materials remains a huge challenge both for the fluorescent materials or the phosphorescent materials. In general, the current organic light-emitting diodes adopting blue fluorescent materials are of high reliability. Nonetheless, the internal quantum efficiency of most current blue fluorescent materials is limited by quantum statistics to a maximum of 25%, resulting in low overall luminescence efficiency. And the wide emission spectrum and the poor color purity are not conducive to high-quality display. The synthesis of these fluorescent material is also relatively complex, which is not conducive to large-scale mass production. Meanwhile, their device stability still requires further improvement. Therefore, the development of highly efficient and stable blue fluorescent materials is an urgent challenge to be solved in the industry.

In the prior art, the light-emitting layer of the blue organic electroluminescent element uses a host-guest doping structure. The existing blue host materials are mainly based on fused ring derivatives of anthracene, as described in patents CN1914293B, CN102448945B, U.S.2015287928A1, etc. However, these compounds suffer from insufficient luminescence efficiency and brightness, as well as poor device lifetime. The blue guest compounds in the prior art can be aryl vinylamines (WO04013073, WO04016575, WO04018587). However, the poor thermal stability and easy decomposition of these compounds lead to poor device lifetime, which is the most significant drawback of OLED materials in the industry today. In order to achieve high efficiency and long life of blue devices, a blue host material with steric hindrance is disclosed in patent WO2017010489A1 et al., which can obtain high luminescence efficiency and device lifetime.

In order to further improve the efficiency and lifetime of the blue devices, further material improvements are still needed. For blue OLED, the host material is the key material that determines its lifetime, and high-performance blue host materials have long been the focus of material development.

### SUMMARY

In one aspect, the present disclosure provides an organic mixture comprising a first organic compound H₁ and a second organic compound H₂, where
1) ΔE_{ST}(H₁)≥0.6 eV, E_{X}-T₁(H₁)≥0.6 eV; and/or
2) ΔE_{ST}(H₂)≥0.6 eV, E_{X}-T₁(H₂)≥0.6 eV; and
3) LUMO(H₂)≤LUMO(H₁)+0.10 eV;

Where ΔE_{ST} equals E_{S1}-E_{T1}, E_{X} equals min(|HOMO(H₁)-LUMO(H₂)|, |HOMO(H₂)-LUMO(H₁)|), E_{S1} stands for the single energy level, E_{T1} stands for the triplet energy level, HOMO stands for the highest occupied molecular orbital energy level, LUMO stands for the lowest unoccupied molecular orbital energy level.

In addition or alternatively, the organic mixture further comprises at least one other organic functional material, the at least one other organic functional material may be selected from the group consisting of a hole-injection material (HIM), a hole-transport material (HTM), a hole-blocking material (HBM), an electron-injection material (EIM), an electron-transport material (ETM), an electron-blocking material (EBM), an organic host material (Host), a singlet emitting material (fluorescent emitting material), a triplet emitting material (phosphorescent emitting material), a thermally activated delayed fluorescence material (TADF material), and an organic dye.

In another aspect, the present disclosure also provides a formulation comprising at least one organic mixture as described herein and at least one organic solvent.

In yet another aspect, the present disclosure further provides an organic electronic device comprising at least one organic mixture as described herein.

Beneficial effects: in the organic mixture matching certain energy level structure as described herein, a transition excited state may form between two organic compounds (i.e., the first organic compound H₁ and the second organic compound H₂). The exciton energy of the transition excited state is significantly higher than that of the T₁ excited states of both organic compounds, so that a conventional exciplex cannot form. However, the organic mixture as described herein is based on fused-ring compounds and has a special energy level structure that facilitates the formation of efficient transition excited states. When the energy differences between the transition excited state and the S₁ states of the two organic compounds are small enough, the energy transfer from the transition excited state to the S₁ state can occur quickly; or, in the presence of an additional light emitter (dopant), such a transition excited state can rapidly transfer energy to the S₁ state of the dopant. The organic electroluminescent element comprising such organic mixtures as light-emitting layer materials might have high luminescence efficiency and long device lifetime. One possible reason may be that the ratio of S₁ to T₁ in the transition excited states is higher than 1:3.

### DETAILED DESCRIPTION OF THE EMBODIMENTS

The present disclosure provides an organic mixture and the applications thereof in the organic electronic devices. The present disclosure may be embodied in many different forms and is not limited to the embodiments described herein. Rather, these embodiments are provided so that the understanding of the invention of the present disclosure will be more thorough.

Unless otherwise defined, all technical and scientific terms used herein have the same meaning as commonly understood by one of ordinary skill in the art belonging to the present disclosure. The terms used herein in the description of the present disclosure are used only for the purpose of describing specific embodiments and are not intended to be limiting of the present disclosure. As used herein, the term “and/or” includes any and all combinations of one or more of the relevant listed items.

As used herein, the terms “host material”, “matrix material” have the same meaning, and they are interchangeable with each other.

As used herein, the terms “formulation”, “printing ink”, and “inks” have the same meaning, and they are interchangeable with each other.

In one aspect, the present disclosure provides an organic mixture comprising a first organic compound H₁ and a second organic compound H₂, where
1) ΔE_{ST}(H₁)≥0.6 eV, E_{X}-T₁(H₁)≥0.6 eV; and/or
2) ΔE_{ST}(H₂)≥0.6 eV, E_{X}-T₁(H₂)≥0.6 eV; and
3) LUMO(H₂)≤LUMO(H₁)+0.10 eV;

Where ΔE_{ST} equals E_{S1}-E_{T1}, E_{X} equals min (|HOMO(H₁)-LUMO(H₂)|, |HOMO(H₂)-LUMO(H₁)|), E_{S1} stands for the singlet energy level, E_{T1} stands for the triplet energy level, HOMO stands for the highest occupied molecular orbital energy level, LUMO stands for the lowest unoccupied molecular orbital energy level.

In some embodiments, ΔE_{ST}(H₁)≥0.7 eV; in some embodiments, ΔE_{ST}(H₁)≥0.8 eV; in some embodiments, ΔE_{ST}(H₁)≥0.9 eV; in some embodiments, ΔE_{ST}(H₁)≥1.0 eV.

In some embodiments, ΔE_{ST}(H₂)≥0.7 eV; in some embodiments, ΔE_{ST}(H₂)≥0.8 eV; in some embodiments, ΔE_{ST}(H₂)≥0.9 eV; in some embodiments, ΔE_{ST}(H₂)≥1.0 eV.

In some embodiments, E_{X}-T₁(H₁)≥0.7 eV; in some embodiments, E_{X}-T₁(H₁)≥0.8 eV; in some embodiments, E_{X}-T₁(H₁)≥0.9 eV; in some embodiments, E_{X}-T₁(H₁)≥1.0 eV.

In some embodiments, E_{X}-T₁(H₂)≥0.7 eV; in some embodiments, E_{X}-T₁(H₂)≥0.8 eV; in some embodiments, E_{X}-T₁(H₂)≥0.9 eV; in some embodiments, E_{X}-T₁(H₂)≥1.0 eV.

In some embodiments, LUMO(H₂)≤LUMO(H₁)+0.08 eV; in some embodiments, LUMO(H₂)≤LUMO(H₁)+0.06 eV; in some embodiments, LUMO(H₂)≤LUMO(H₁)+0.04 eV; in some embodiments, LUMO(H₂)≤LUMO(H₁)+0.02 eV; in some embodiments, LUMO(H₂)≤LUMO(H₁).

In the organic mixture as described herein, |E_{X}-E_{S1}(H₁)|≤0.4 eV or |E_{X}-E_{S1}(H₂)|≤0.4 eV.

In some embodiments, |E_{X}-S₁(H₁)|≤0.3 eV or |E_{X}-S₁(H₂)|≤0.3 eV; in some embodiments, |E_{X}-S₁(H₁)|≤0.2 eV or |E_{X}-S₁(H₂)|≤0.2 eV; in some embodiments, |E_{X}-S₁(H₁)|≤0.1 eV or |E_{X}-S₁(H₂)|≤0.1 eV; in some embodiments, |E_{X}-S₁(H₁)|≤0.05 eV or |E_{X}-S₁(H₂)|≤0.05 eV.

In the organic mixture matching a certain energy level structure as described herein, a transition excited state may form between two organic compounds (i.e., the first organic compound H₁ and the second organic compound H₂). The exciton energy of the transition excited state is significantly higher than that of the T₁ excited states of both organic compounds. When the energy differences between the transition excited state and the S₁ states of the two organic compounds are small enough, the energy transfer from the transition excited state to the S₁ state can occur quickly.

In the organic mixture as described herein, both the first organic compound H₁ and the second organic compound H₂ are independently selected from the group consisting of a substituted/unsubstituted aromatic or heteroaromatic group containing 5 to 40 ring atoms, an aryloxy or heteroaryloxy group containing 5 to 40 ring atoms, and any combination thereof.

In some embodiments, in the organic mixture as described herein, both the first organic compound H₁ and the second organic compound H₂ are independently selected from the group consisting of a substituted/unsubstituted aromatic or heteroaromatic group containing 5 to 30 ring atoms, an aryloxy or heteroaryloxy group containing 5 to 30 ring atoms, and any combination thereof.

In some embodiments, in the organic mixture as described herein, both the first organic compound H₁ and the second organic compound H₂ are independently selected from the group consisting of a substituted/unsubstituted aromatic or heteroaromatic group containing 5 to 20 ring atoms, an aryloxy or heteroaryloxy group containing 5 to 20 ring atoms, and any combination thereof.

One or more H atoms of the foregoing groups can be further substituted with D atoms.

In some embodiments, the aromatic ring system contains 5 to 15 carbon atoms, more preferably 5 to 10 carbon atoms, the heteroaromatic ring system contains 2 to 15 carbon atoms, more preferably 2 to 10 carbon atoms, together with at least one heteroatom, while the total number of carbon atoms and heteroatoms is at least 4. The heteroatoms are preferably selected from Si, N, P, O, S and/or Ge, particularly preferably from Si, N, P, O and/or S, and more particularly preferably from N, O, or S.

The term “aromatic ring system” or “aromatic group” refers to a hydrocarbon group consisting of an aromatic ring, including monocyclic groups and polycyclic systems. The term “heteroaromatic ring system” or “heteroaromatic group” refers to a hydrocarbon group (containing a heteroatom) consisting of at least one heteroaromatic ring, including monocyclic groups and polycyclic systems. The polycyclic systems contain two or more rings, in which two carbon atoms are shared by two adjacent rings, i. e., fused rings. Specifically, at least one of the rings in the polycyclic rings are aromatic or heteroaromatic. For the purposes of the present disclosure, the aromatic ring groups or heteroaromatic groups comprise not only aromatic or heteroaromatic systems, but also a plurality of aromatic or heteroaromatic groups are interconnected by short non-aromatic units (for example by <10% of non-H atoms, more specifically 5% of non-H atoms, such as C, N or O atoms). Therefore, systems such as 9,9′-spirobifluorene, 9,9-diaryl fluorene, triarylamine, diaryl ethers, and other systems, should also be considered as aromatic groups for the purpose of this disclosure.

Specifically, examples of the aromatic groups include benzene, naphthalene, anthracene, phenanthrene, perylene, naphthacene, pyrene, benzpyrene, triphenylene, acenaphthene, fluorene, spirofluorene, and derivatives thereof.

Specifically, examples of heteroaromatic groups include: furan, benzofuran, dibenzofuran, thiophene, benzothiophene, dibenzothiophene, pyrrole, pyrazole, triazole, imidazole, oxazole, oxadiazole, thiazole, tetrazole, indole, carbazole, pyrroloimidazole, pyrrolopyrrole, thienopyrrole, thienothiophene, furopyrrole, furofuran, thienofuran, benzisoxazole, benzoisothiazole, benzimidazole, pyridine, pyrazine, pyridazine, pyrimidine, triazine, quinoline, isoquinoline, phenanthroline, quinoxaline, phenanthridine, primidine, quinazoline, quinazolinone, and derivatives thereof.

In the organic mixture as described herein, the second organic compound H₂ contains an electron-accepting group.

In some embodiments, the second organic compound H₂ contains two electron-accepting groups.

In some embodiments, the second organic compound H₂ contains three electron-accepting groups.

In some embodiments, the second organic compound H₂ contains more than three electron-accepting groups.

The above-mentioned electron-accepting group may be selected from F, a cyano group, or one of the following groups:

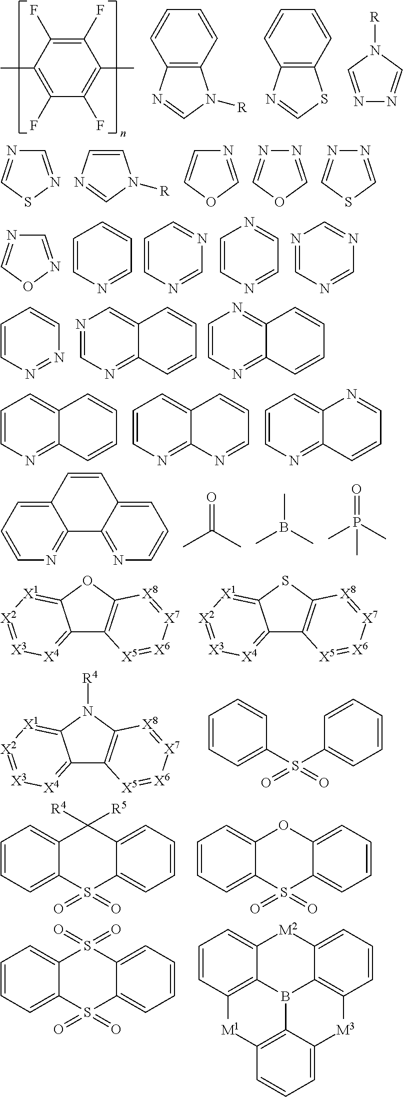

Where n is an integer from 1 to 3; each of X¹ to X⁸ is independently selected from CR or N, and at least one N; M¹, M², M³ are independently selected from N(R), C(R)₂, Si(R)₂, O, C═N(R), C═C(R)₂, P(R), P(═O)R, S, S═O, SO₂, or null; each of R⁴ and R⁵ is independently selected from the following structures: H, D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ substituted/unsubstituted branched/cyclic silyl group, a C₁-C₂₀ substituted ketone group, a C₂-C₂₀ alkoxycarbonyl group, a C₇-C₂₀ aryloxycarbonyl group, a cyano group (—CN), a carbamoyl group (—C(═O)NH₂), a haloformyl group (—C(═O)—X where X represents a halogen atom), a formyl group (—C(═O)—H), an isocyano group, an isocyanate group, a thiocyanate/isothiocyanate group, a hydroxyl group, a nitro group, a CF₃ group, Cl, Br, F, a cross-linkable group, a substituted/unsubstituted aromatic or heteroaromatic group containing 5 to 40 ring atoms, an aryloxy or heteroaryloxy group containing 5 to 40 ring atoms, and any combination thereof, where one or more R⁴-R⁵ can form a monocyclic or polycyclic aliphatic or aromatic ring system with each other and/or with the rings bonded to the R⁴-R⁵. Each R is independently selected from a substituted/unsubstituted alkyl group containing 1 to 30 carbon atoms, a substituted/unsubstituted cycloalkyl group containing 3 to 30 carbon atoms, or a substituted/unsubstituted aromatic hydrocarbon or aromatic heterocyclic group containing 5 to 60 ring atoms.

In some embodiments, the second organic compound H₂ contains F.

In some embodiments, the second organic compound H₂ contains CN.

In the organic mixture as described herein, the first organic compound H₁ or the second organic compound H₂ is selected from the following structures:

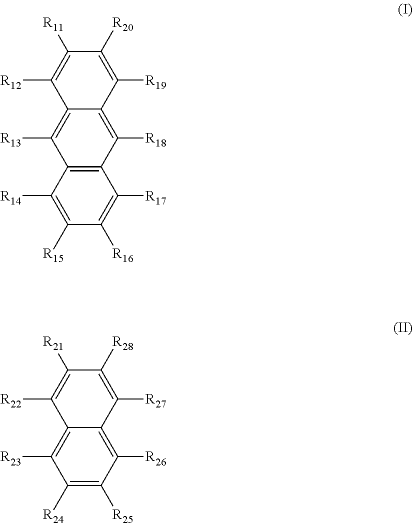

Where R₁₁ to R₂₈ are independently selected from the group consisting of —H, —D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ substituted/unsubstituted branched/cyclic silyl group, a C₁-C₂₀ substituted ketone group, a C₂-C₂₀ alkoxycarbonyl group, a C₇-C₂₀ aryloxycarbonyl group, a cyano group (—CN), a carbamoyl group (—C(═O)NH₂), a haloformyl group (—C(═O)—X where X represents a halogen atom), a formyl group (—C(═O)—H), an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic or heteroaromatic group containing 5 to 40 ring atoms, an aryloxy or heteroaryloxy group containing 5 to 40 ring atoms, and any combination thereof, where one or more R₁₁-R₂₈ can form a monocyclic or polycyclic aliphatic or aromatic ring system with each other and/or with the rings bonded to the R₁₁-R₂₈.

In some embodiments, R₁₁ to R₂₈ are independently selected from the group consisting of —H, —D, a C₁-C₁₀ linear alkyl group, a C₁-C₁₀ alkoxy group, a C₁-C₁₀ thioalkoxy group, a C₃-C₁₀ branched/cyclic alkyl group, a C₃-C₁₀ branched/cyclic alkoxy group, a C₃-C₁₀ branched/cyclic thioalkoxy group, a C₃-C₁₀ substituted/unsubstituted branched/cyclic silyl group, a C₁-C₁₀ substituted ketone group, a C₂-C₁₀ alkoxycarbonyl group, a C₇-C₁₀ aryloxycarbonyl group, a cyano group (—CN), a carbamoyl group (—C(═O)NH₂), a haloformyl group (—C(═O)—X where X represents a halogen atom), a formyl group (—C(═O)—H), an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic or heteroaromatic group containing 5 to 20 ring atoms, an aryloxy or heteroaryloxy group containing 5 to 20 ring atoms, and any combination thereof, where one or more R₁₁-R₂₈ can form a monocyclic or polycyclic aliphatic or aromatic ring system with each other and/or with the rings bonded to the R₁₁-R₂₈.

In the organic mixture of formula (I) or (II), R₁₁ to R₂₈ are independently selected from one or combinations of more than one structures of the following Table 1:

TABLE 1

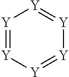

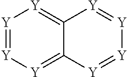

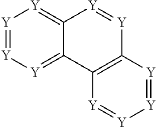

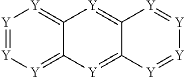

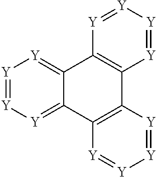

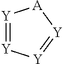

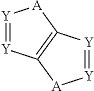

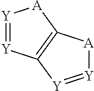

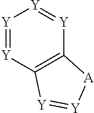

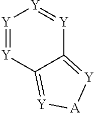

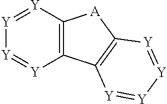

Where each Y is CR₇₀₁ or N; each A is selected from O, S, CR₇₀₂R₇₀₃, or NR₇₀₄; R₇₀₁ to R ₇₀₄ in multiple occurrences may be sites linked to other groups, or be independently selected from the group consisting of —H, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ substituted/unsubstituted branched/cyclic silyl group, a C₁-C₂₀ substituted ketone group, a C₂-C₂₀ alkoxycarbonyl group, a C₇-C₂₀ aryloxycarbonyl group, a cyano group (—CN), a carbamoyl group (—C(═O)NH₂), a haloformyl group (—C(═O)—X where X represents a halogen atom), a formyl group (—C(═O)—H), an isocyano group, an isocyanate group, a thiocyanate, an isothiocyanate group, a hydroxyl group, a nitro group, a CF₃ group, Cl, Br, F, a cross-linkable group, a substituted/unsubstituted aromatic or heteroaromatic group containing 5 to 40 ring atoms, an aryloxy or heteroaryloxy group containing 5 to 40 ring atoms, and any combination thereof, where one or more R₇₀₁-R₇₀₄ can form a monocyclic or polycyclic aliphatic or aromatic ring system with each other and/or with the rings bonded to the R₇₀₁-R₇₀₄.

In some embodiments, each Y is CR₇₀₁.

In some embodiments, at least one Y in each structure is N atom.

In some embodiments, at least two Ys in each structure are N atoms.

In some embodiments, at least three Ys in each structure are N atoms.

More preferably, R₇₀₁ to R₇₀₄ in multiple occurrences can be sites linked to other groups, are independently selected from the group consisting of a single bond, —H, a C₁-C₁₀ linear alkyl group, a C₁-C₁₀ alkoxy group, a C₁-C₁₀ thioalkoxy group, a C₃-C₁₀ branched/cyclic alkyl group, a C₃-C₁₀ branched/cyclic alkoxy group, a C₃-C₁₀ branched/cyclic thioalkoxy group, a C₃-C₁₀ substituted/unsubstituted branched/cyclic silyl group, a C₁-C₁₀ substituted ketone group, a C₂-C₁₀ alkoxycarbonyl group, a C₇-C₁₀ aryloxycarbonyl group, a cyano group (—CN), a carbamoyl group (—C(═O)NH₂), a haloformyl group (—C(═O)—X where X represents a halogen atom), a formyl group (—C(═O)—H), an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic or heteroaromatic group containing 5 to 20 ring atoms, an aryloxy or heteroaryloxy group containing 5 to 20 ring atoms, and any combination thereof, where one or more R₇₀₁-R₇₀₄ can form a monocyclic or polycyclic aliphatic or aromatic ring system with each other and/or with the rings bonded to the R₇₀₁-R₇₀₄. One or more H atoms of the foregoing groups can be further substituted with D atoms.

In the organic mixture as described herein, the first compound H₁ or the second compound H₂ is selected from the following structures:

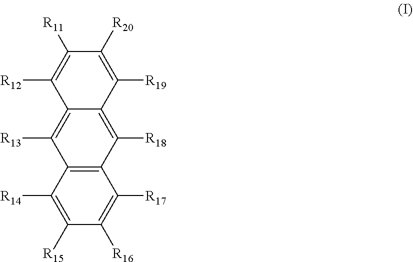

Where R₁₁ to R₂₀ are independently selected from the group consisting of —H, —D, a C₁-C₂₀ linear alkyl group, a C₁-C₂₀ alkoxy group, a C₁-C₂₀ thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ substituted/unsubstituted branched/cyclic silyl group, a C₁-C₂₀ substituted ketone group, a C₂-C₂₀ alkoxycarbonyl group, a C₇-C₂₀ aryloxycarbonyl group, a cyano group (—CN), a carbamoyl group (—C(═O)NH₂), a haloformyl group (—C(═O)—X where X represents a halogen atom), a formyl group (—C(═O)—H), an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic or heteroaromatic group containing 5 to 40 ring atoms, an aryloxy or heteroaryloxy group containing 5 to 40 ring atoms, and any combination thereof, where one or more R₁₁-R₂₀ can form a monocyclic or polycyclic aliphatic or aromatic ring system with each other and/or with the rings bonded to the R₁₁-R₂₀.

Preferably, R₁₁ to R₂₀ are independently selected from the group consisting of —H, —D, a C₁-C₁₀ linear alkyl group, a C₁-C₁₀ alkoxy group, a C₁-C₁₀ thioalkoxy group, a C₃-C₁₀ branched/cyclic alkyl group, a C₃-C₁₀ branched/cyclic alkoxy group, a C₃-C₁₀ branched/cyclic thioalkoxy group, a C₃-C₁₀ substituted/unsubstituted branched/cyclic silyl group, a C₁-C₁₀ substituted ketone group, a C₂-C₁₀ alkoxycarbonyl group, a C₇-C₁₀ aryloxycarbonyl group, a cyano group (—CN), a carbamoyl group (—C(═O)NH₂), a haloformyl group (—C(═O)—X where X represents a halogen atom), a formyl group (—C(═O)—H), an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic or heteroaromatic group containing 5 to 20 ring atoms, an aryloxy or heteroaryloxy group containing 5 to 20 ring atoms, and any combination thereof, where one or more R₁₁-R₂₀ can form a monocyclic or polycyclic aliphatic or aromatic ring system with each other and/or with the rings bonded to the R₁₁-R₂₀.

In some embodiments, each of R₁₁, R₁₂, R₁₄, R₁₅, R₁₆, R₁₇, R₁₉, R₂₀ is H or D; each of R₁₃ and R₁₈ is independently selected from one or combinations of more than one structures of the Table 1.

In the organic mixture as described herein, the first organic compound H₁ or the second organic compound H₂ is selected from the following structures:

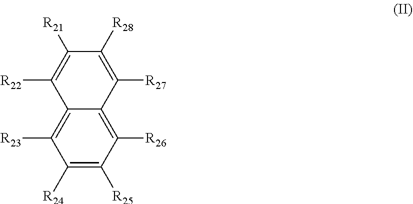

Where R₂₁ to R₂₈ are independently selected from the group consisting of —H, —D, a C₁-C₂₀ linear alkyl group, a C₁-C₂₀ alkoxy group, a C₁-C₂₀ thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ substituted/unsubstituted branched/cyclic silyl group, a C₁-C₂₀ substituted ketone group, a C₂-C₂₀ alkoxycarbonyl group, a C₇-C₂₀ aryloxycarbonyl group, a cyano group (—CN), a carbamoyl group (—C(═O)NH₂), a haloformyl group (—C(═O)—X where X represents a halogen atom), a formyl group (—C(═O)—H), an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic or heteroaromatic group containing 5 to 40 ring atoms, an aryloxy or heteroaryloxy group containing 5 to 40 ring atoms, and any combination thereof, where one or more R₂₁-R₂₈ can form a monocyclic or polycyclic aliphatic or aromatic ring system with each other and/or with the rings bonded to the R₂₁-R₂₈.

Preferably, R₂₁ to R₂₈ are independently selected from the group consisting of —H, —D, a C₁-C₁₀ linear alkyl group, a C₁-C₁₀ alkoxy group, a C₁-C₁₀ thioalkoxy group, a C₃-C₁₀ branched/cyclic alkyl group, a C₃-C₁₀ branched/cyclic alkoxy group, a C₃-C₁₀ branched/cyclic thioalkoxy group, a C₃-C₁₀ substituted/unsubstituted branched/cyclic silyl group, a C₁-C₁₀ substituted ketone group, a C₂-C₁₀ alkoxycarbonyl group, a C₇-C₁₀ aryloxycarbonyl group, a cyano group (—CN), a carbamoyl group (—C(═O)NH₂), a haloformyl group (—C(═O)—X where X represents a halogen atom), a formyl group (—C(═O)—H), an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic or heteroaromatic group containing 5 to 20 ring atoms, an aryloxy or heteroaryloxy group containing 5 to 20 ring atoms, and any combination thereof, where one or more R₂₁-R₂₈ can form a monocyclic or polycyclic aliphatic or aromatic ring system with each other and/or with the rings bonded to the R₂₁-R₂₈.

In some embodiments, each of R₂₁, R₂₄, R₂₅, R₂₈ is H or D; each of R₂₂, R₂₃, R₂₆, R₂₇ is independently selected from one or combinations of more than one structures of the Table 1.

In some embodiments, R₁₁ to R₂₈ are independently selected from one of the following structures:

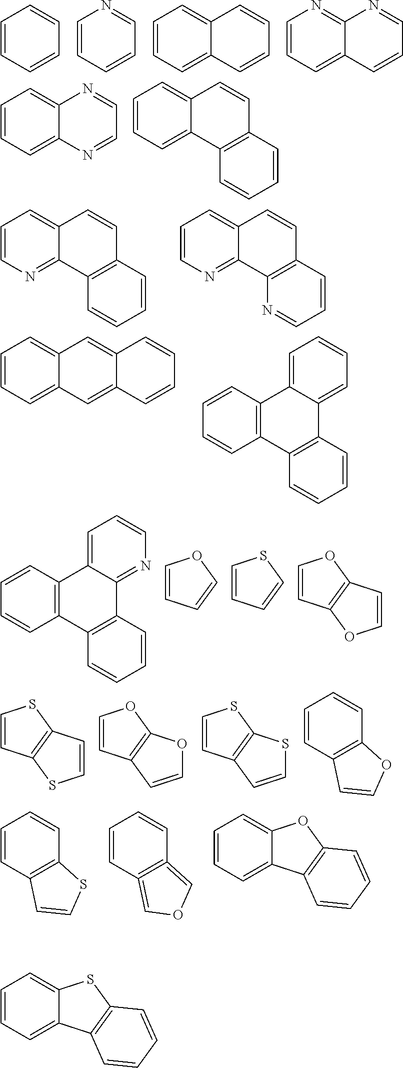

In some embodiments, the first organic compound H₁ and the second organic compound H₂ of the organic mixture are formed type I heterojunction energy structure.

In some embodiments, the first organic compound H₁ and the second organic compound H₂ of the organic mixture are formed type II heterojunction energy structure.

In some embodiments, in the organic mixture as described herein, the molar ratio of the first organic compound H₁ to the second organic compound H₂ is from 2: 8 to 8: 2; preferably from 3: 7 to 7: 3; more preferably from 4: 6 to _{6: 4.}

In some embodiments, the first organic compound H₁ and/or the second organic compound H₂ of the organic mixture have relatively large oscillator strength (f(S₁), f(S₂), f(S₃)), preferably at least one of them>0.05, more preferably at least one of them>0.10, and most preferably at least one of them>0.15. The oscillator strength may be obtained by quantum chemical simulation as described in the following embodiments.

In some embodiments, in the organic mixture as described herein, the first organic compound H₁ and/or the second organic compound H₂ have an oscillator strength (f(S₁))≥0.05, preferably ≥0.10, more preferably ≥0.15, and most preferably ≥0.18.

In some embodiments, in the first organic compound H₁ and the second organic compound H₂ of the organic mixture as described herein, at least one of them has a glass transition temperature (Tg) ≥100° C.; in some embodiments, at least one of them has a Tg ≥120° C.; in some embodiments, at least one of them has a Tg ≥140° C., in some embodiments, at least one of them has a Tg ≥160° C.; in some embodiments, at least one of them has a Tg ≥180° C.

In some embodiments, in the organic mixture as described herein, at least one of the first organic compound H₁ or the second organic compound H₂ is partially deuterated, preferably of 10% or more total H, more preferably of 20% or more total H, further preferably of 30% or more total H, and most preferably of 40% or more total H.

In some embodiments, the first organic compound H₁ and the second organic compound H₂ of the organic mixture as described herein are both small molecular materials.

It is an object of the present disclosure to provide a material for the evaporation-based OLEDs.

In some embodiments, the organic mixture material as described herein is used for evaporation-based OLEDs. For this purpose, the first organic compound H₁ and the second organic compound H₂ of the organic mixture as described herein have a molecular weight ≤1000 g/mol, preferably ≤900 g/mol, more preferably ≤850 g/mol, further preferably ≤800 g/mol, and most preferably ≤700 g/mol.

In some embodiments, in the organic mixture as described herein, the molecular weight difference between the first organic compound H₁ and the second organic compound H₂≤100 Daltons; preferably ≤60 Daltons, more preferably ≤30 Daltons.

In some embodiments, in the organic mixture as described herein, the sublimation temperature difference between the first organic compound H₁ and the second organic compound H₂≤30 K; preferably ≤20 K; more preferably ≤10 K.

Another object of the present disclosure is to provide a material for the printed OLEDs.

For this purpose, at least one of, preferably both the first organic compound H₁ and the second organic compound H₂ of the organic mixture as described herein has a molecular weight ≥700 g/mol, preferably ≥800 g/mol, more preferably ≥900 g/mol, further preferably ≥1000 g/mol, and most preferably ≥1100 g/mol.

For the evaporation-based OLED, the co-host applied in the form of a Premix requires that the two host materials have similar chemical properties or physical properties, such as molecular weight and sublimation temperature. Meanwhile in solution-processed OLEDs, two host materials with different properties may improve film-forming performance, thereby improving the performance of the devices. The properties mentioned can be glass transition temperature, molecular volumes, etc, in addition to molecular weight, sublimation temperature. For these purposes, some embodiments of the organic mixture as described herein include:
1) The molecular weight difference between the first organic compound H₁ and the second organic compound H₂≥120 g/mol, preferably ≥140 g/mol, more preferably ≥160 g/mol, and most preferably ≥180 g/mol.
2) The sublimation temperature difference between the first organic compound H₁ and the second organic compound H₂≤80 K, preferably ≤75 K, more preferably ≤70 K, and most preferably ≤60 K.
3) The glass transition temperature difference between the first organic compound H₁ and the second organic compound H₂≤45 K, preferably ≤40 K, more preferably ≤30 K, and most preferably ≤35 K.
4) The molecular volume difference between the first organic compound H₁ and the second organic compound H₂≥20%, preferably ≥30%, more preferably ≥40%, and most preferably ≥45%.

In some embodiments, at least one of, preferably both the first organic compound H₁ and the second organic compound H₂ of the organic mixture as described herein has a solubility of ≥2 mg/ml in toluene at 25° C., preferably ≥3 mg/ml, more preferably ≥4 mg/ml, and most preferably ≥5 mg/ml.

In some embodiments, at least one of the first organic compound H₁ or the second organic compound H₂ in the organic mixture as described herein has a solubility of ≥6 mg/ml in toluene at 25° C., preferably ≥8 mg/ml, more preferably ≥10 mg/ml, and most preferably ≥15 mg/ml. Better yet, both the first organic compound H₁ and the second organic compound H₂ have solubilities of ≥6 mg/ml in toluene at 25° C., preferably ≥8 mg/ml, more preferably ≥10 mg/ml, and most preferably ≥15 mg/ml.

The term “small molecule” herein refers to a molecule that is no one of following: a polymer, an oligomer, a dendrimer, or a blend. In particular, there are no repeating structures in the small molecule. The molecular weight of a small molecule ≤3000 g/mol, preferably ≤2000 g/mol, and most preferably ≤1500 g/mol.

The term of polymer comprises homopolymer, copolymer, and block copolymer. Also as used herein, the term of polymer comprises dendrimer. For the synthesis and application of the dendrimers please refer to [Dendrimers and Dendrons, Wiley-VCH Verlag GmbH & Co. KGaA, 2002, Ed. George R. Newkome, Charles N. Moorefield, Fritz Vogtle.].

The term “conjugated polymer” refers to a polymer with backbone mainly comprising sp2 hybrid orbitals of C atoms, well-known example are polyacetylene and poly(phenylene vinylene). The C atoms on the backbones can also be substituted with other non-C atoms. Moreover, the above-mentioned structure should still be considered as a conjugated polymer when the sp2 hybridization on the backbone is interrupted by natural defects. Also as used herein, the backbone of the conjugated polymer comprises aryl amines, aryl phosphines and other heteroarmotics, organom etallic complexes, etc.

Examples of specific first organic compound H₁ of the organic mixture as described herein are as follows, but not limited thereto.

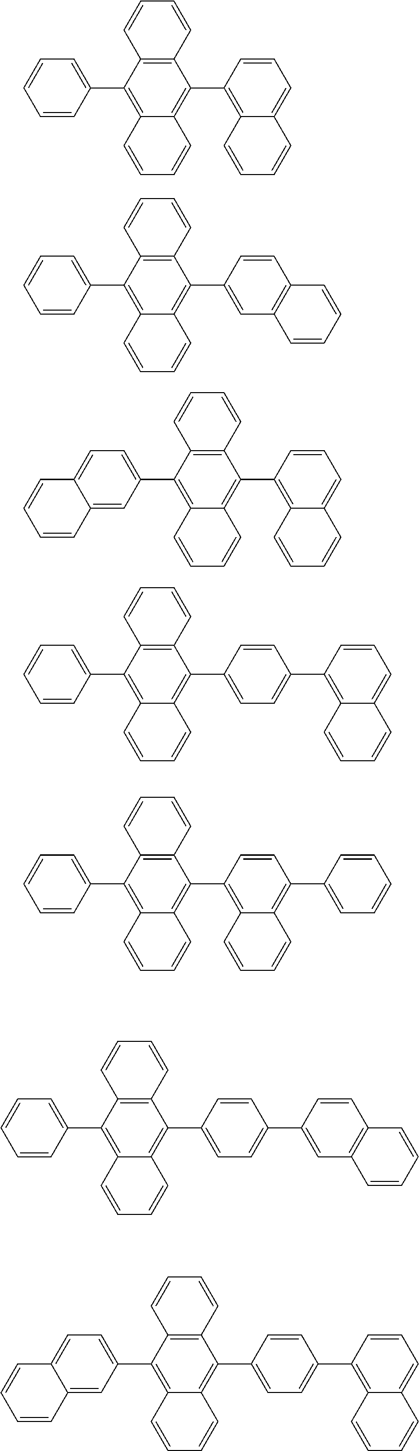

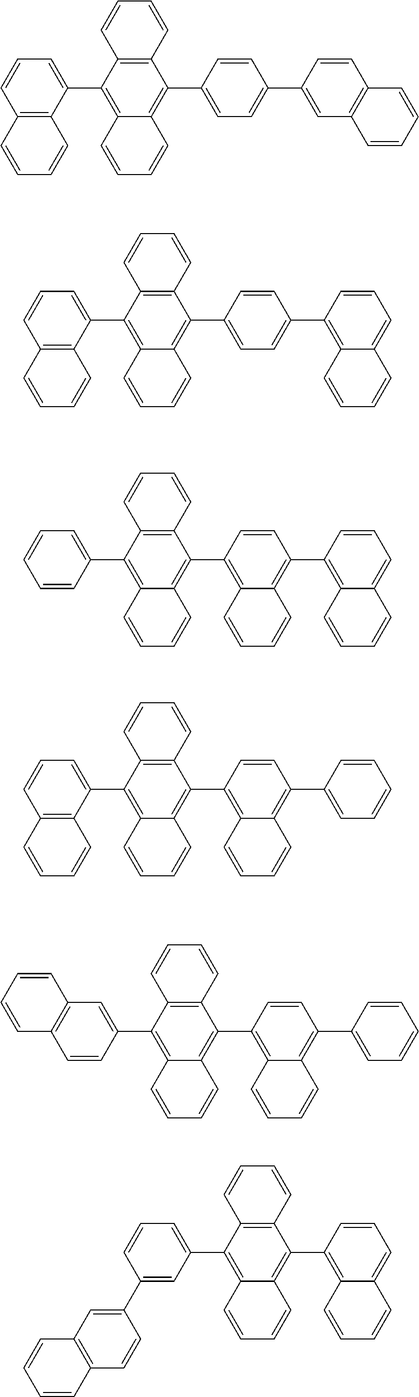

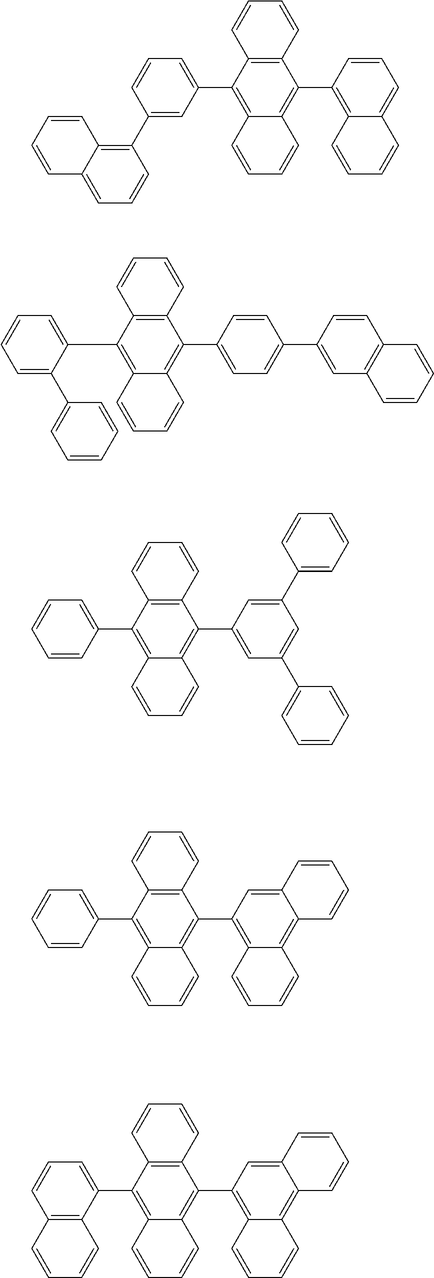

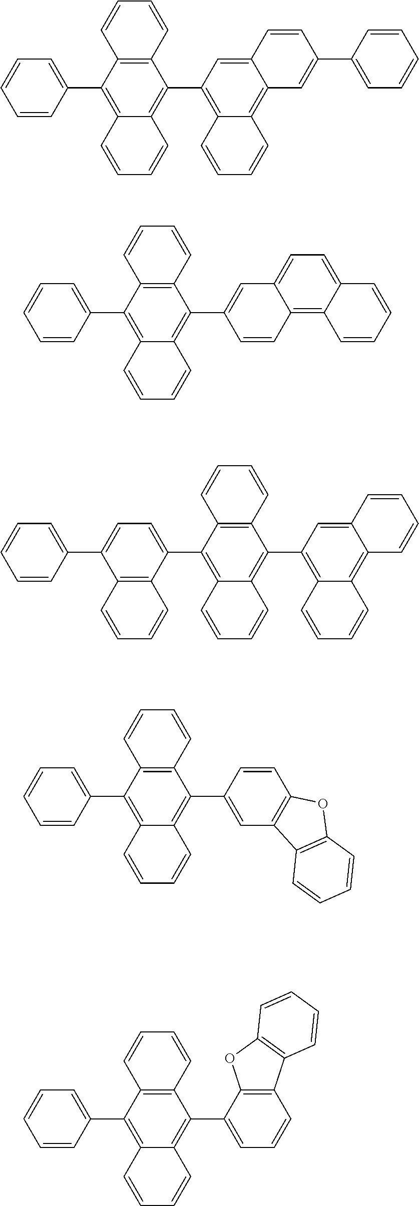

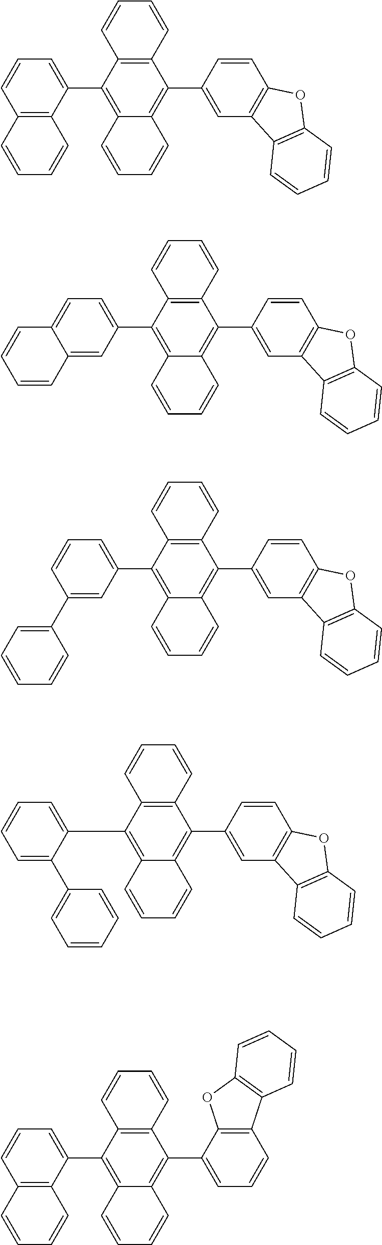 [SMILES: c1ccc2c(-c3c4ccccc4c(-c4ccc5oc6ccccc6c5c4)c4ccccc34)cccc2c1 ; c1ccc2cc(-c3c4ccccc4c(-c4ccc5oc6ccccc6c5c4)c4ccccc34)ccc2c1 ; c1ccc(-c2cccc(-c3c4ccccc4c(-c4ccc5oc6ccccc6c5c4)c4ccccc34)c2)cc1 ; c1ccc(-c2ccccc2-c2c3ccccc3c(-c3ccc4oc5ccccc5c4c3)c3ccccc23)cc1 ; c1ccc2c(-c3c4ccccc4c(-c4cccc5c4oc4ccccc45)c4ccccc34)cccc2c1]

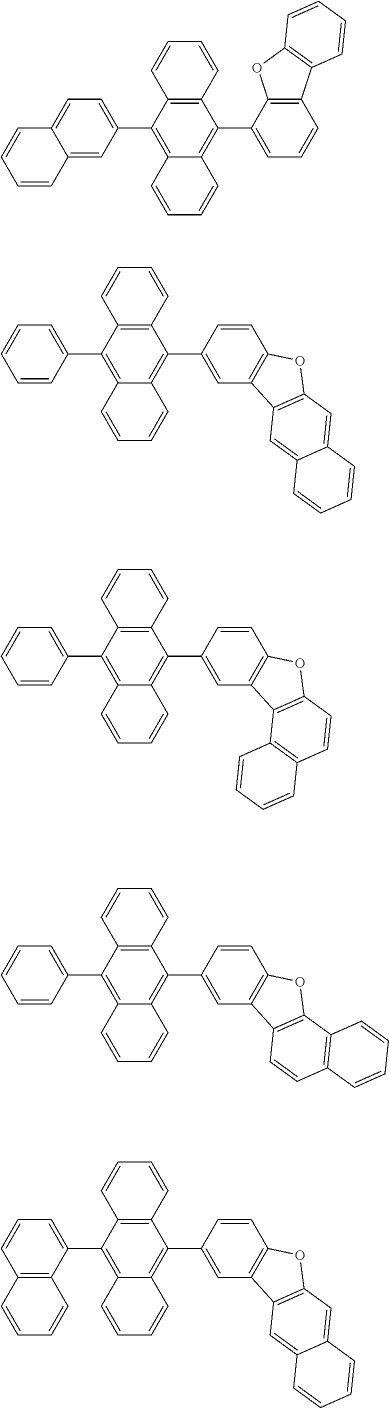 [SMILES: c1ccc2cc(-c3c4ccccc4c(-c4cccc5c4oc4ccccc45)c4ccccc34)ccc2c1 ; c1ccc(-c2c3ccccc3c(-c3ccc4oc5cc6ccccc6cc5c4c3)c3ccccc23)cc1 ; c1ccc(-c2c3ccccc3c(-c3ccc4oc5ccc6ccccc6c5c4c3)c3ccccc23)cc1 ; c1ccc(-c2c3ccccc3c(-c3ccc4oc5c6ccccc6ccc5c4c3)c3ccccc23)cc1 ; c1ccc2cc3c(cc2c1)oc1ccc(-c2c4ccccc4c(-c4cccc5ccccc45)c4ccccc24)cc13]

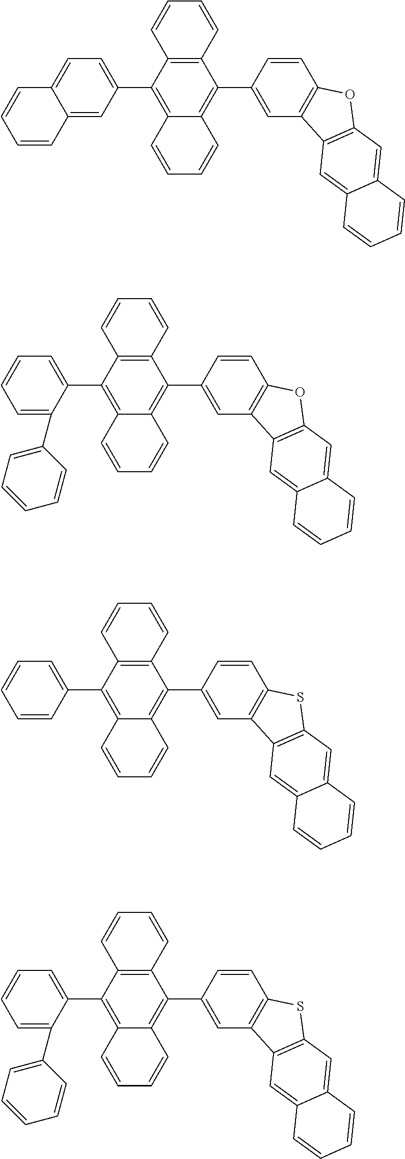 [SMILES: c1ccc2cc(-c3c4ccccc4c(-c4ccc5oc6cc7ccccc7cc6c5c4)c4ccccc34)ccc2c1 ; c1ccc(-c2ccccc2-c2c3ccccc3c(-c3ccc4oc5cc6ccccc6cc5c4c3)c3ccccc23)cc1 ; c1ccc(-c2c3ccccc3c(-c3ccc4sc5cc6ccccc6cc5c4c3)c3ccccc23)cc1 ; c1ccc(-c2ccccc2-c2c3ccccc3c(-c3ccc4sc5cc6ccccc6cc5c4c3)c3ccccc23)cc1]

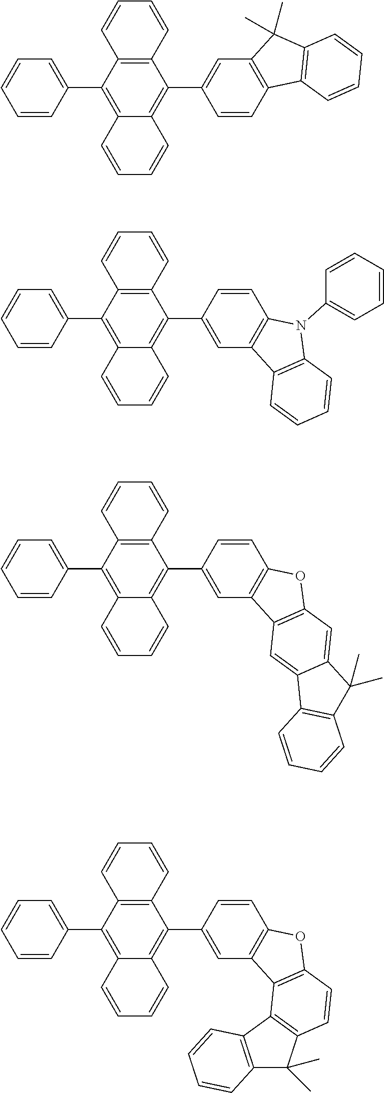

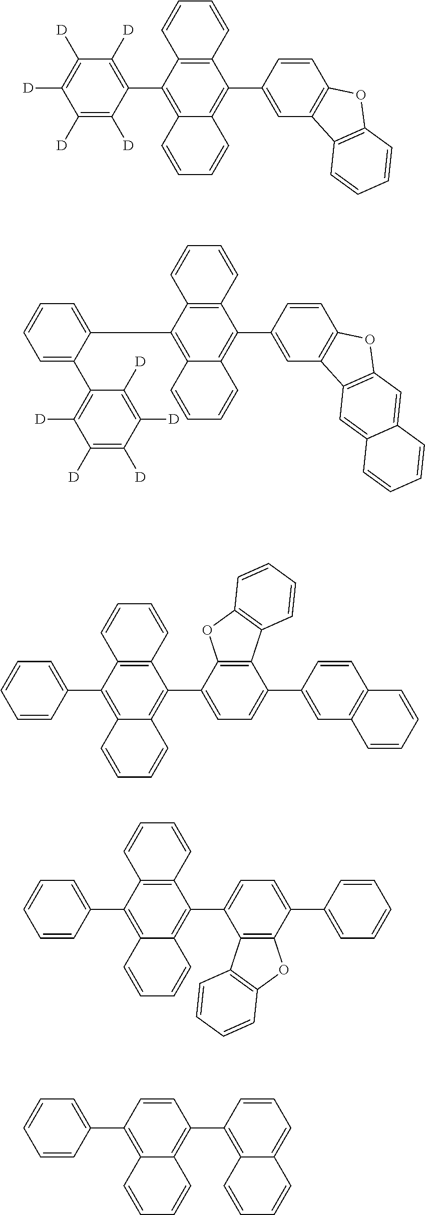

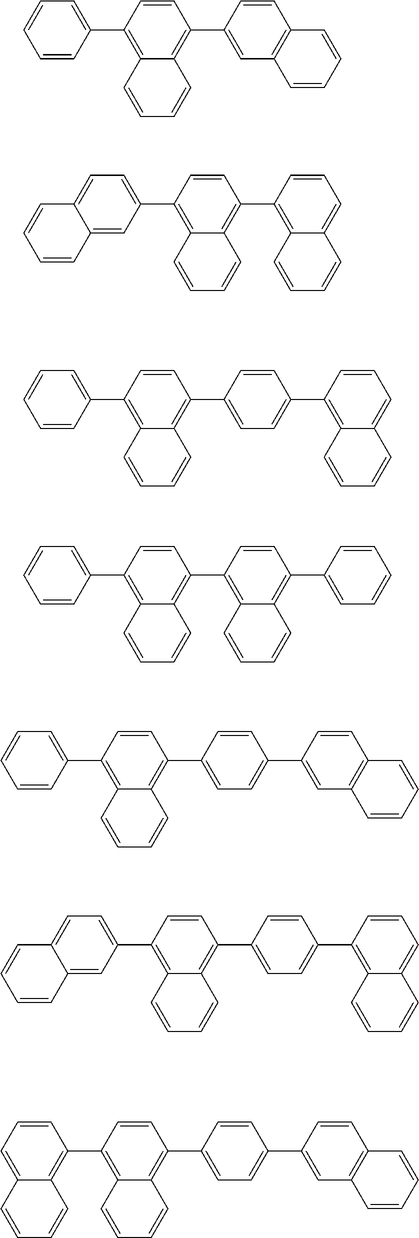

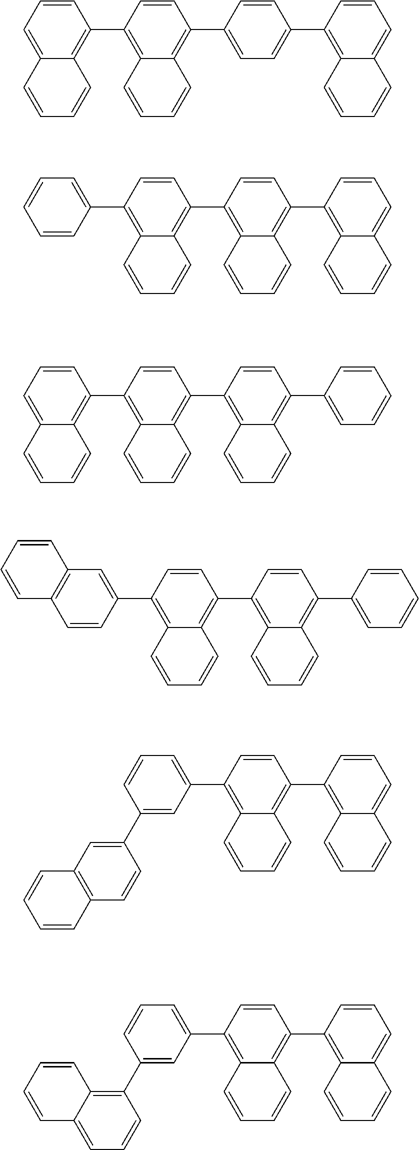

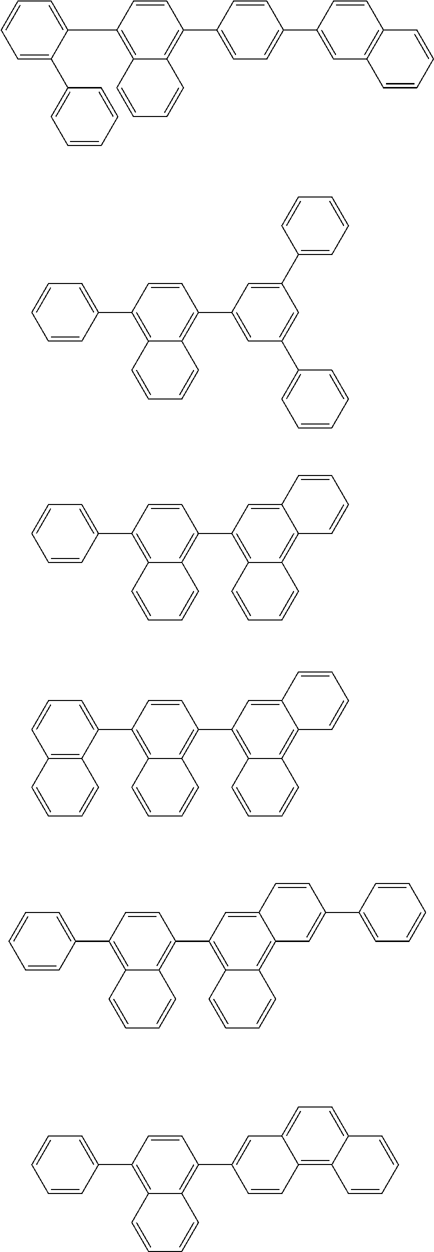

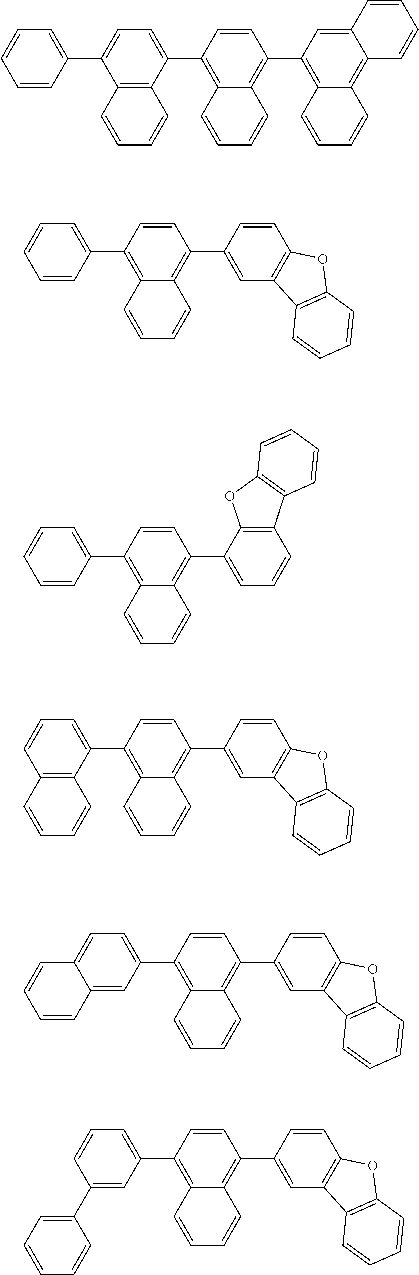

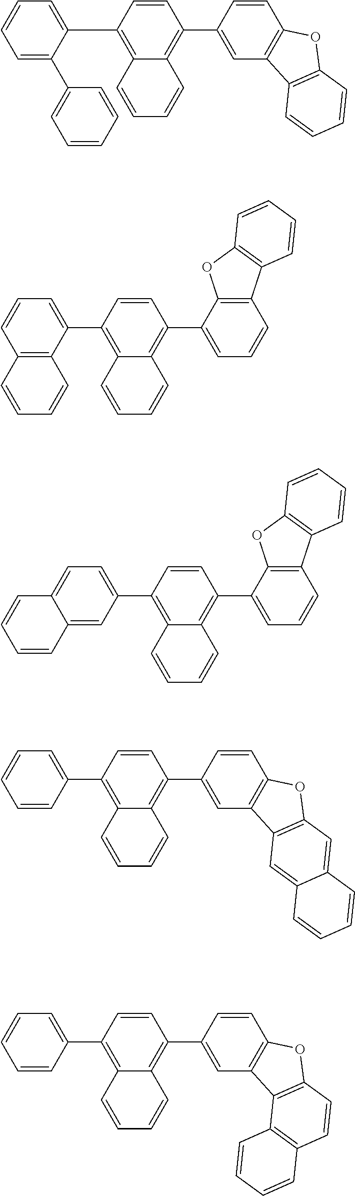 [SMILES: c1ccc(-c2ccccc2-c2ccc(-c3ccc4oc5ccccc5c4c3)c3ccccc23)cc1 ; c1ccc2c(-c3ccc(-c4cccc5c4oc4ccccc45)c4ccccc34)cccc2c1 ; c1ccc2cc(-c3ccc(-c4cccc5c4oc4ccccc45)c4ccccc34)ccc2c1 ; c1ccc(-c2ccc(-c3ccc4oc5cc6ccccc6cc5c4c3)c3ccccc23)cc1 ; c1ccc(-c2ccc(-c3ccc4oc5ccc6ccccc6c5c4c3)c3ccccc23)cc1]

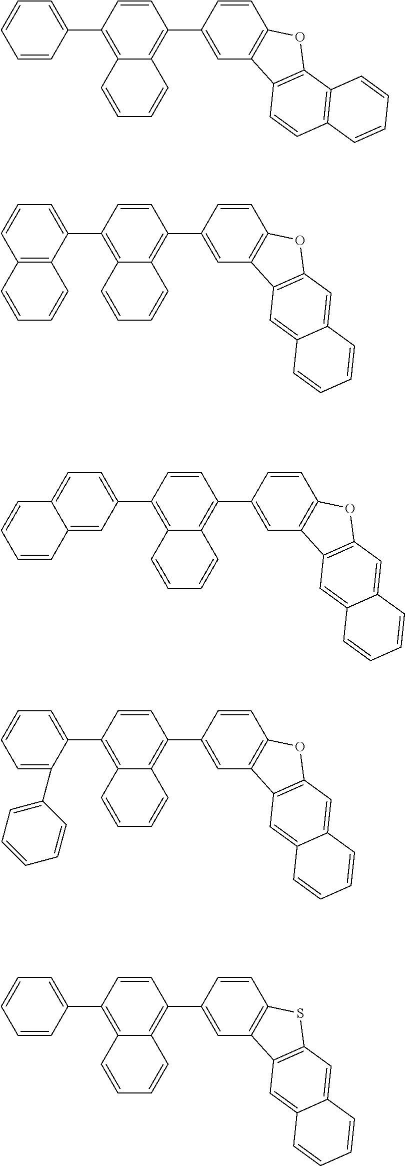 [SMILES: c1ccc(-c2ccc(-c3ccc4oc5c6ccccc6ccc5c4c3)c3ccccc23)cc1 ; c1ccc2cc3c(cc2c1)oc1ccc(-c2ccc(-c4cccc5ccccc45)c4ccccc24)cc13 ; c1ccc2cc(-c3ccc(-c4ccc5oc6cc7ccccc7cc6c5c4)c4ccccc34)ccc2c1 ; c1ccc(-c2ccccc2-c2ccc(-c3ccc4oc5cc6ccccc6cc5c4c3)c3ccccc23)cc1 ; c1ccc(-c2ccc(-c3ccc4sc5cc6ccccc6cc5c4c3)c3ccccc23)cc1]

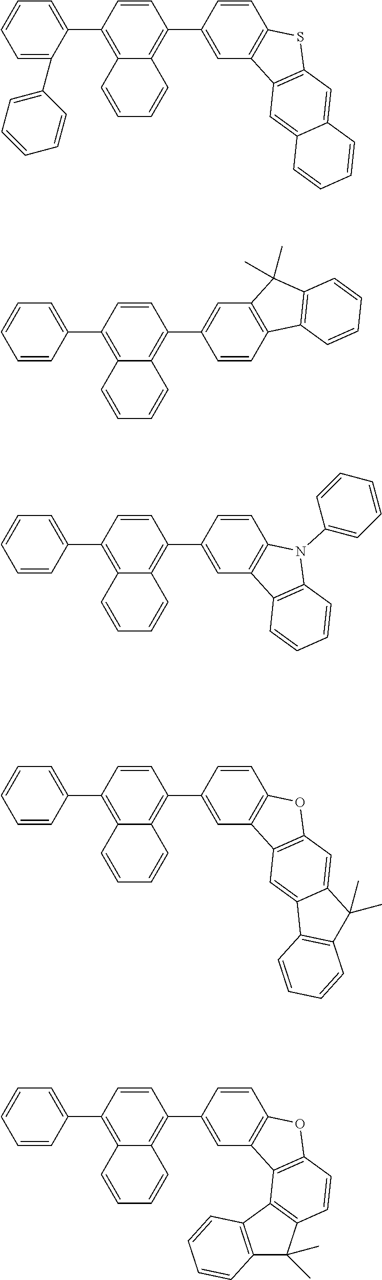

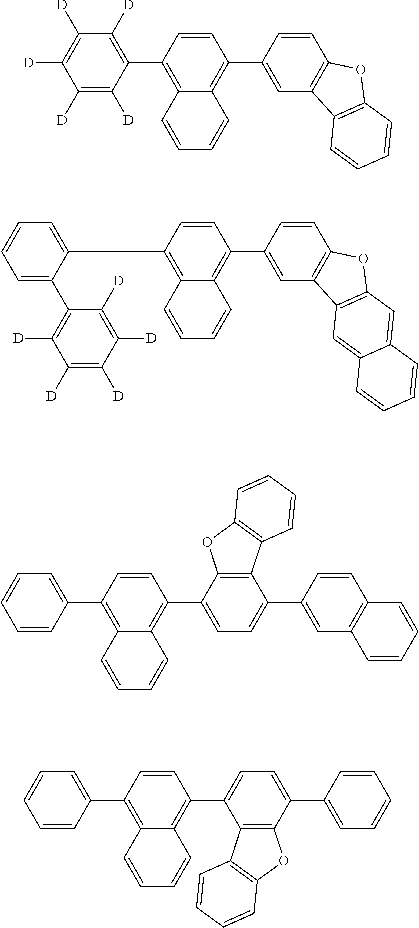 [SMILES: [2H]c1c([2H])c([2H])c(-c2ccc(-c3ccc4oc5ccccc5c4c3)c3ccccc23)c([2H])c1[2H] ; [2H]c1c([2H])c([2H])c(-c2ccccc2-c2ccc(-c3ccc4oc5cc6ccccc6cc5c4c3)c3ccccc23)c([2H])c1[2H] ; c1ccc(-c2ccc(-c3ccc(-c4ccc5ccccc5c4)c4c3oc3ccccc34)c3ccccc23)cc1 ; c1ccc(-c2ccc(-c3ccc(-c4ccccc4)c4oc5ccccc5c34)c3ccccc23)cc1]

Examples of specific second organic compound H₂ of the organic mixture as described herein are as follows, but not limited thereto.

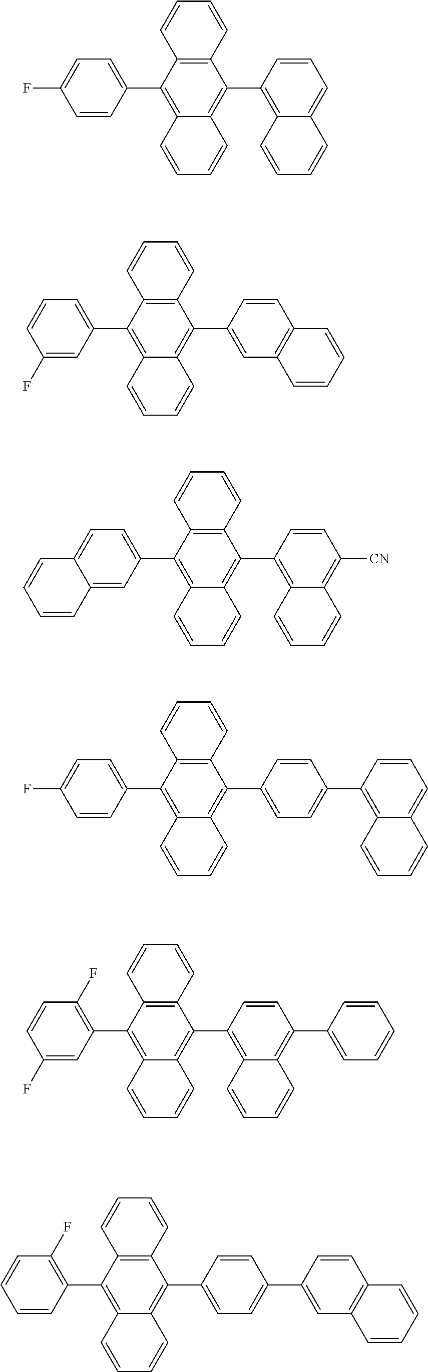 [SMILES: Fc1ccc(-c2c3ccccc3c(-c3cccc4ccccc34)c3ccccc23)cc1 ; Fc1cccc(-c2c3ccccc3c(-c3ccc4ccccc4c3)c3ccccc23)c1 ; N#Cc1ccc(-c2c3ccccc3c(-c3ccc4ccccc4c3)c3ccccc23)c2ccccc12 ; Fc1ccc(-c2c3ccccc3c(-c3ccc(-c4cccc5ccccc45)cc3)c3ccccc23)cc1 ; Fc1ccc(F)c(-c2c3ccccc3c(-c3ccc(-c4ccccc4)c4ccccc34)c3ccccc23)c1 ; Fc1ccccc1-c1c2ccccc2c(-c2ccc(-c3ccc4ccccc4c3)cc2)c2ccccc12]

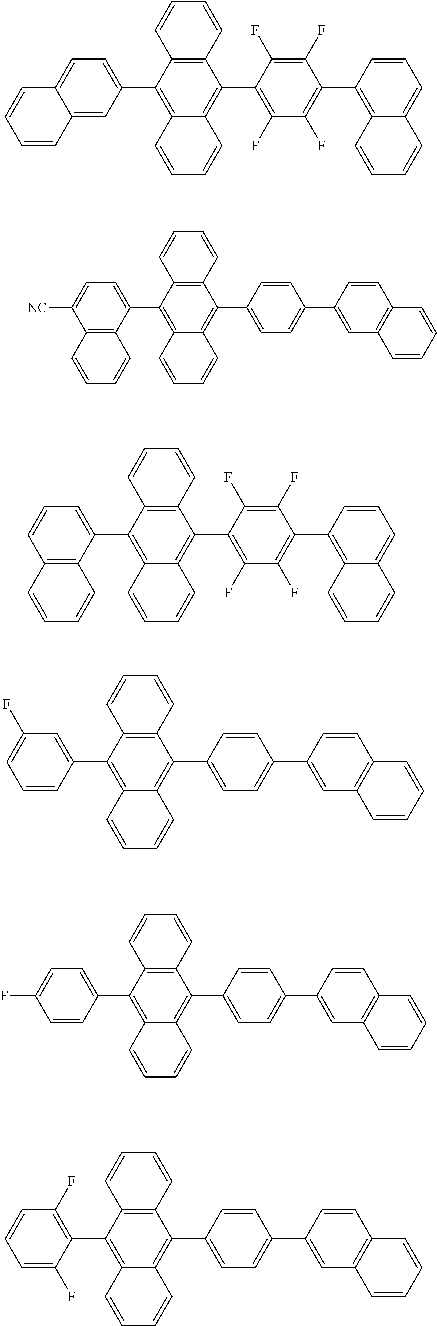 [SMILES: Fc1c(F)c(-c2c3ccccc3c(-c3ccc4ccccc4c3)c3ccccc23)c(F)c(F)c1-c1cccc2ccccc12 ; N#Cc1ccc(-c2c3ccccc3c(-c3ccc(-c4ccc5ccccc5c4)cc3)c3ccccc23)c2ccccc12 ; Fc1c(F)c(-c2c3ccccc3c(-c3cccc4ccccc34)c3ccccc23)c(F)c(F)c1-c1cccc2ccccc12 ; Fc1cccc(-c2c3ccccc3c(-c3ccc(-c4ccc5ccccc5c4)cc3)c3ccccc23)c1 ; Fc1ccc(-c2c3ccccc3c(-c3ccc(-c4ccc5ccccc5c4)cc3)c3ccccc23)cc1 ; Fc1cccc(F)c1-c1c2ccccc2c(-c2ccc(-c3ccc4ccccc4c3)cc2)c2ccccc12]

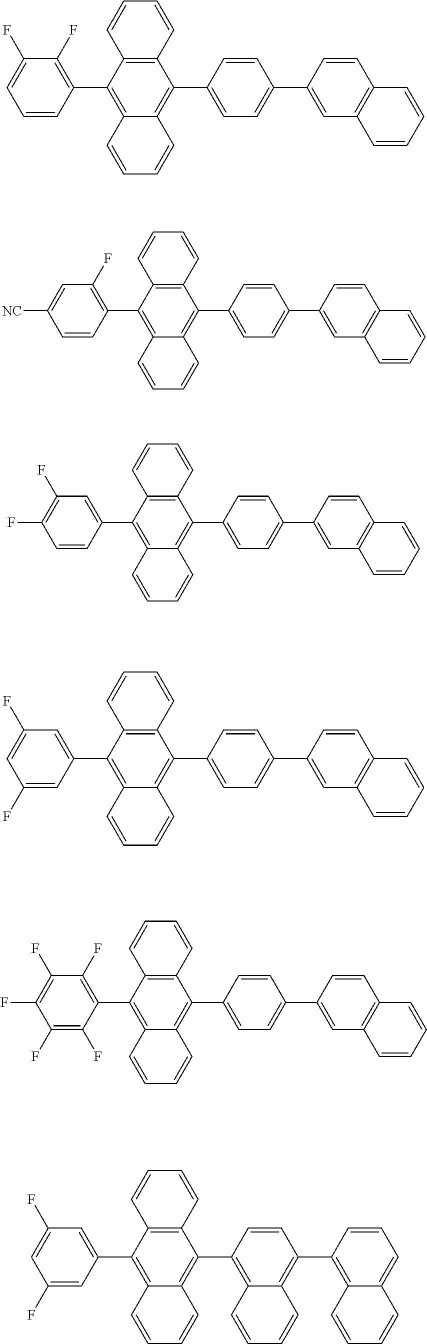 [SMILES: Fc1cccc(-c2c3ccccc3c(-c3ccc(-c4ccc5ccccc5c4)cc3)c3ccccc23)c1F ; N#Cc1ccc(-c2c3ccccc3c(-c3ccc(-c4ccc5ccccc5c4)cc3)c3ccccc23)c(F)c1 ; Fc1ccc(-c2c3ccccc3c(-c3ccc(-c4ccc5ccccc5c4)cc3)c3ccccc23)cc1F ; Fc1cc(F)cc(-c2c3ccccc3c(-c3ccc(-c4ccc5ccccc5c4)cc3)c3ccccc23)c1 ; Fc1c(F)c(F)c(-c2c3ccccc3c(-c3ccc(-c4ccc5ccccc5c4)cc3)c3ccccc23)c(F)c1F ; Fc1cc(F)cc(-c2c3ccccc3c(-c3ccc(-c4cccc5ccccc45)c4ccccc34)c3ccccc23)c1]

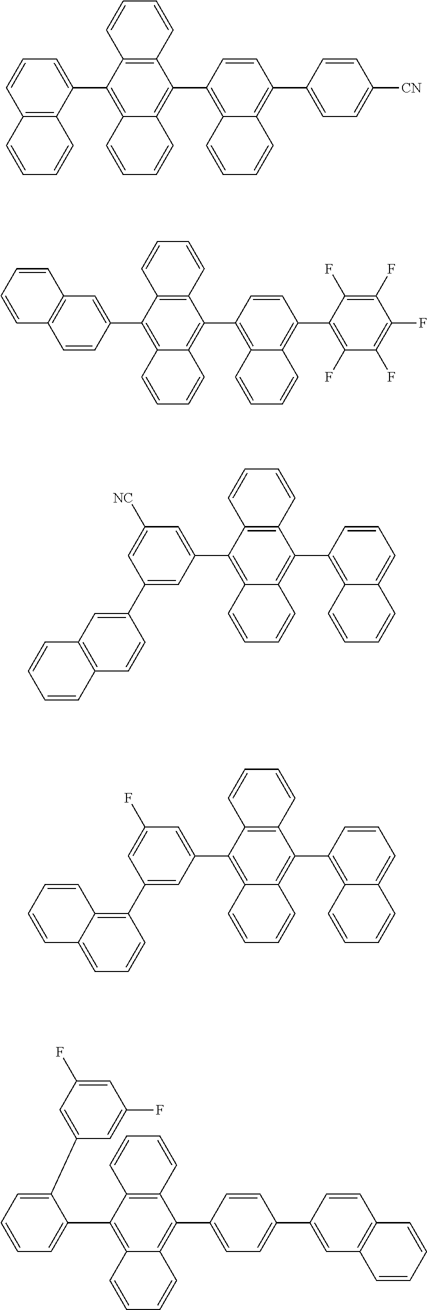 [SMILES: N#Cc1ccc(-c2ccc(-c3c4ccccc4c(-c4cccc5ccccc45)c4ccccc34)c3ccccc23)cc1 ; Fc1c(F)c(F)c(-c2ccc(-c3c4ccccc4c(-c4ccc5ccccc5c4)c4ccccc34)c3ccccc23)c(F)c1F ; N#Cc1cc(-c2ccc3ccccc3c2)cc(-c2c3ccccc3c(-c3cccc4ccccc34)c3ccccc23)c1 ; Fc1cc(-c2cccc3ccccc23)cc(-c2c3ccccc3c(-c3cccc4ccccc34)c3ccccc23)c1 ; Fc1cc(F)cc(-c2ccccc2-c2c3ccccc3c(-c3ccc(-c4ccc5ccccc5c4)cc3)c3ccccc23)c1]

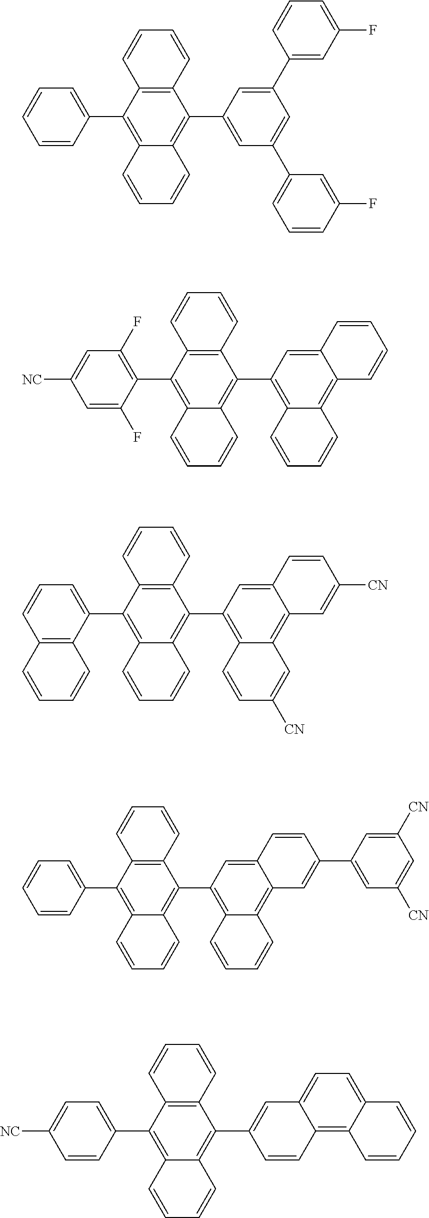 [SMILES: Fc1cccc(-c2cc(-c3cccc(F)c3)cc(-c3c4ccccc4c(-c4ccccc4)c4ccccc34)c2)c1 ; N#Cc1cc(F)c(-c2c3ccccc3c(-c3cc4ccccc4c4ccccc34)c3ccccc23)c(F)c1 ; N#Cc1ccc2cc(-c3c4ccccc4c(-c4cccc5ccccc45)c4ccccc34)c3ccc(C#N)cc3c2c1 ; N#Cc1cc(C#N)cc(-c2ccc3cc(-c4c5ccccc5c(-c5ccccc5)c5ccccc45)c4ccccc4c3c2)c1 ; N#Cc1ccc(-c2c3ccccc3c(-c3ccc4c(ccc5ccccc54)c3)c3ccccc23)cc1]

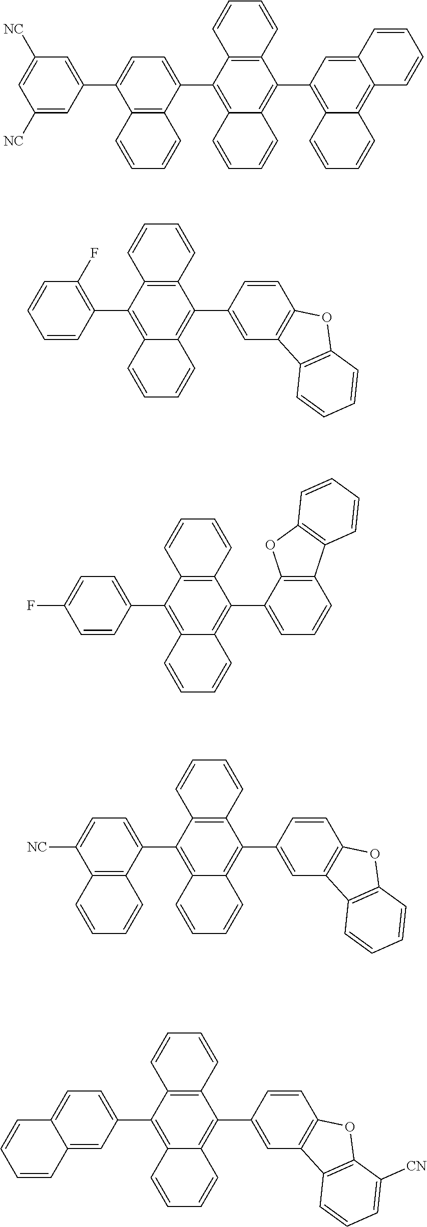 [SMILES: N#Cc1cc(C#N)cc(-c2ccc(-c3c4ccccc4c(-c4cc5ccccc5c5ccccc45)c4ccccc34)c3ccccc23)c1 ; Fc1ccccc1-c1c2ccccc2c(-c2ccc3oc4ccccc4c3c2)c2ccccc12 ; Fc1ccc(-c2c3ccccc3c(-c3cccc4c3oc3ccccc34)c3ccccc23)cc1 ; N#Cc1ccc(-c2c3ccccc3c(-c3ccc4oc5ccccc5c4c3)c3ccccc23)c2ccccc12 ; N#Cc1cccc2c1oc1ccc(-c3c4ccccc4c(-c4ccc5ccccc5c4)c4ccccc34)cc12]

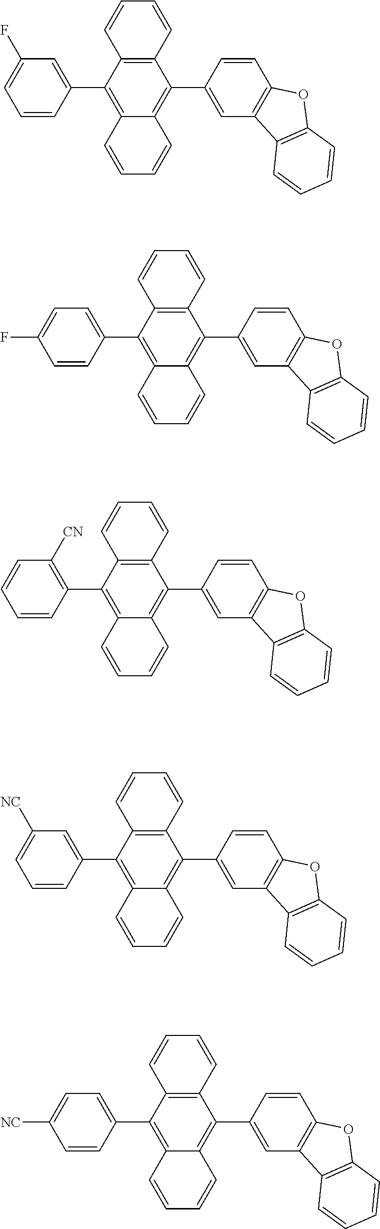 [SMILES: Fc1cccc(-c2c3ccccc3c(-c3ccc4oc5ccccc5c4c3)c3ccccc23)c1 ; Fc1ccc(-c2c3ccccc3c(-c3ccc4oc5ccccc5c4c3)c3ccccc23)cc1 ; N#Cc1ccccc1-c1c2ccccc2c(-c2ccc3oc4ccccc4c3c2)c2ccccc12 ; N#Cc1cccc(-c2c3ccccc3c(-c3ccc4oc5ccccc5c4c3)c3ccccc23)c1 ; N#Cc1ccc(-c2c3ccccc3c(-c3ccc4oc5ccccc5c4c3)c3ccccc23)cc1]

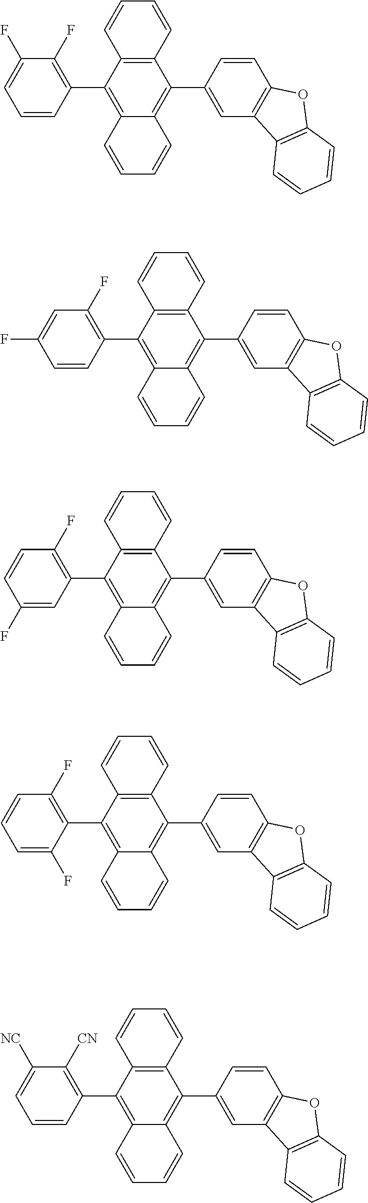 [SMILES: Fc1cccc(-c2c3ccccc3c(-c3ccc4oc5ccccc5c4c3)c3ccccc23)c1F ; Fc1ccc(-c2c3ccccc3c(-c3ccc4oc5ccccc5c4c3)c3ccccc23)c(F)c1 ; Fc1ccc(F)c(-c2c3ccccc3c(-c3ccc4oc5ccccc5c4c3)c3ccccc23)c1 ; Fc1cccc(F)c1-c1c2ccccc2c(-c2ccc3oc4ccccc4c3c2)c2ccccc12 ; N#Cc1cccc(-c2c3ccccc3c(-c3ccc4oc5ccccc5c4c3)c3ccccc23)c1C#N]

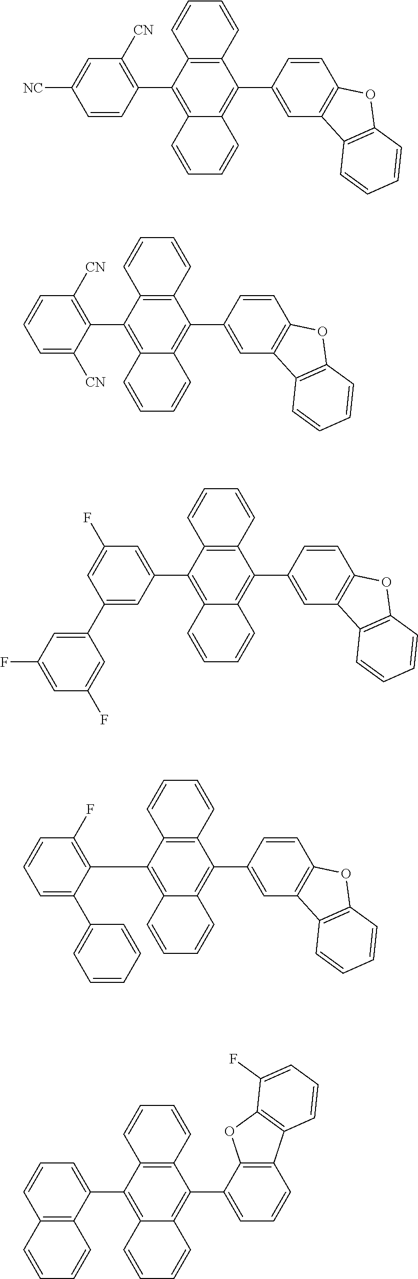 [SMILES: N#Cc1ccc(-c2c3ccccc3c(-c3ccc4oc5ccccc5c4c3)c3ccccc23)c(C#N)c1 ; N#Cc1cccc(C#N)c1-c1c2ccccc2c(-c2ccc3oc4ccccc4c3c2)c2ccccc12 ; Fc1cc(F)cc(-c2cc(F)cc(-c3c4ccccc4c(-c4ccc5oc6ccccc6c5c4)c4ccccc34)c2)c1 ; Fc1cccc(-c2ccccc2)c1-c1c2ccccc2c(-c2ccc3oc4ccccc4c3c2)c2ccccc12 ; Fc1cccc2c1oc1c(-c3c4ccccc4c(-c4cccc5ccccc45)c4ccccc34)cccc12]

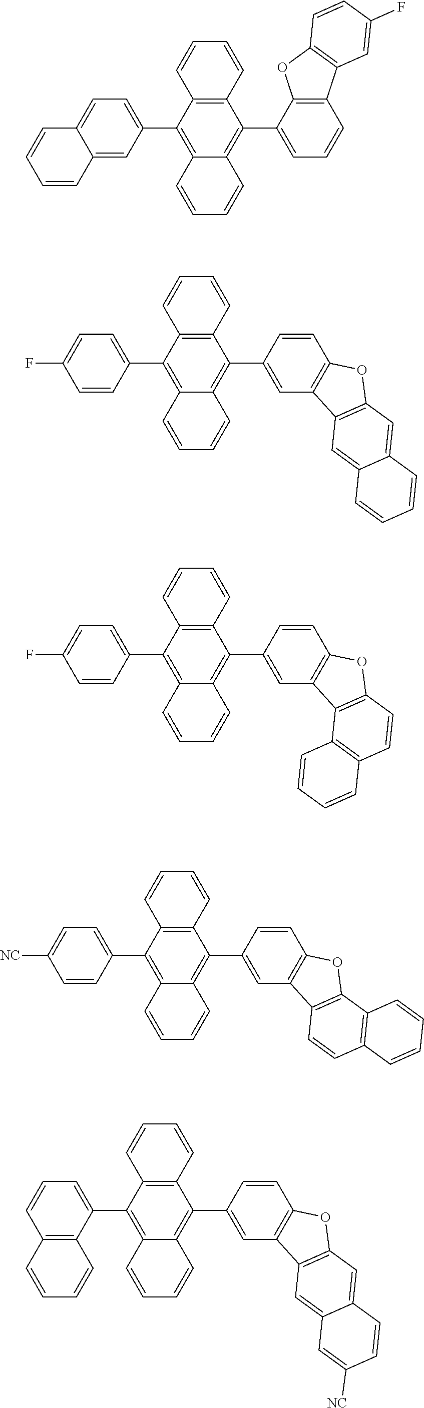 [SMILES: Fc1ccc2oc3c(-c4c5ccccc5c(-c5ccc6ccccc6c5)c5ccccc45)cccc3c2c1 ; Fc1ccc(-c2c3ccccc3c(-c3ccc4oc5cc6ccccc6cc5c4c3)c3ccccc23)cc1 ; Fc1ccc(-c2c3ccccc3c(-c3ccc4oc5ccc6ccccc6c5c4c3)c3ccccc23)cc1 ; N#Cc1ccc(-c2c3ccccc3c(-c3ccc4oc5c6ccccc6ccc5c4c3)c3ccccc23)cc1 ; N#Cc1ccc2cc3oc4ccc(-c5c6ccccc6c(-c6cccc7ccccc67)c6ccccc56)cc4c3cc2c1]

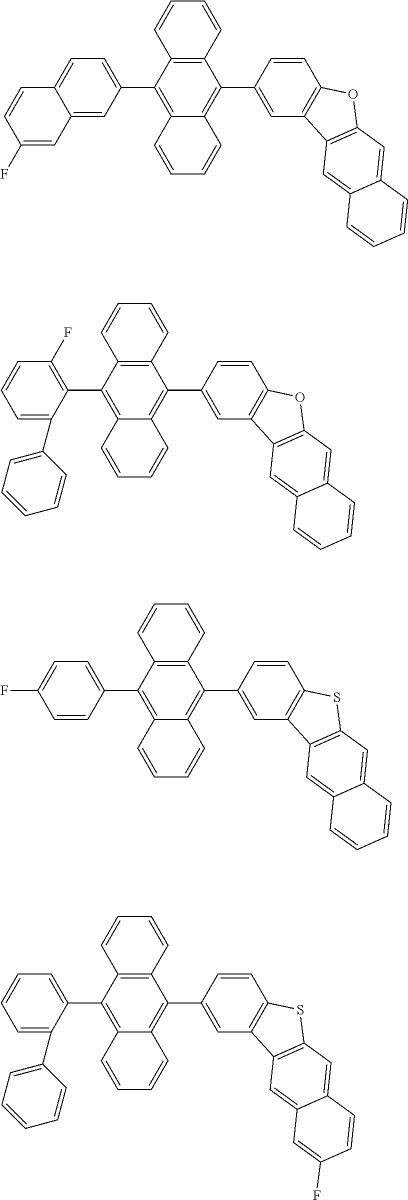 [SMILES: Fc1ccc2ccc(-c3c4ccccc4c(-c4ccc5oc6cc7ccccc7cc6c5c4)c4ccccc34)cc2c1 ; Fc1cccc(-c2ccccc2)c1-c1c2ccccc2c(-c2ccc3oc4cc5ccccc5cc4c3c2)c2ccccc12 ; Fc1ccc(-c2c3ccccc3c(-c3ccc4sc5cc6ccccc6cc5c4c3)c3ccccc23)cc1 ; Fc1ccc2cc3sc4ccc(-c5c6ccccc6c(-c6ccccc6-c6ccccc6)c6ccccc56)cc4c3cc2c1]

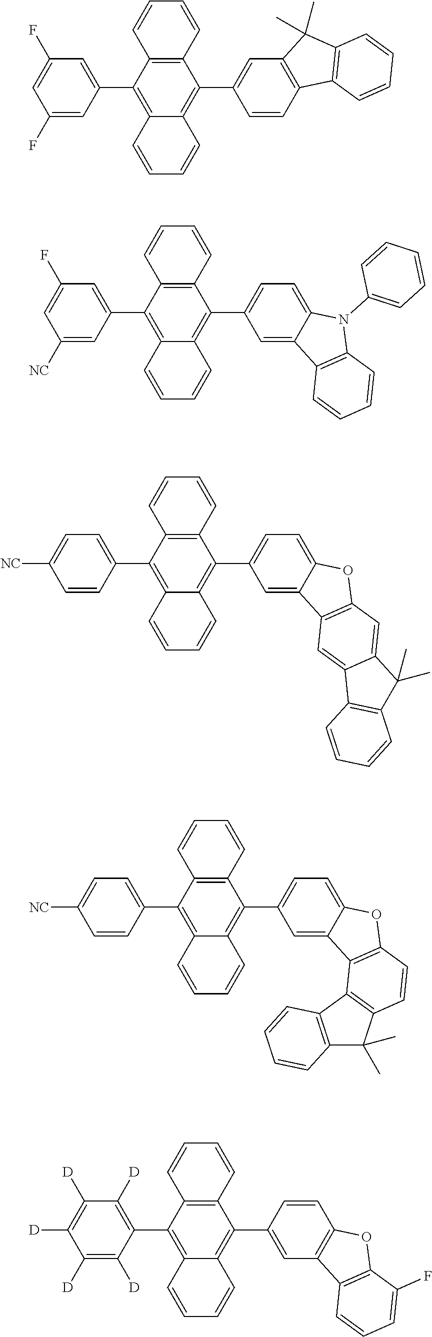 [SMILES: CC1(C)c2ccccc2-c2ccc(-c3c4ccccc4c(-c4cc(F)cc(F)c4)c4ccccc34)cc21 ; N#Cc1cc(F)cc(-c2c3ccccc3c(-c3ccc4c(c3)c3ccccc3n4-c3ccccc3)c3ccccc23)c1 ; CC1(C)c2ccccc2-c2cc3c(cc21)oc1ccc(-c2c4ccccc4c(-c4ccc(C#N)cc4)c4ccccc24)cc13 ; CC1(C)c2ccccc2-c2c1ccc1oc3ccc(-c4c5ccccc5c(-c5ccc(C#N)cc5)c5ccccc45)cc3c21 ; [2H]c1c([2H])c([2H])c(-c2c3ccccc3c(-c3ccc4oc5c(F)cccc5c4c3)c3ccccc23)c([2H])c1[2H]]

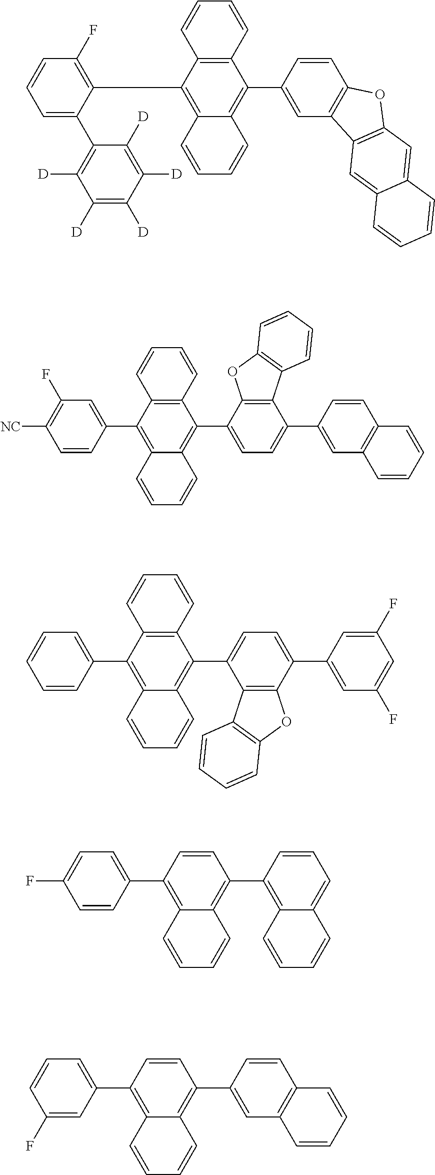 [SMILES: [2H]c1c([2H])c([2H])c(-c2cccc(F)c2-c2c3ccccc3c(-c3ccc4oc5cc6ccccc6cc5c4c3)c3ccccc23)c([2H])c1[2H] ; N#Cc1ccc(-c2c3ccccc3c(-c3ccc(-c4ccc5ccccc5c4)c4c3oc3ccccc34)c3ccccc23)cc1F ; Fc1cc(F)cc(-c2ccc(-c3c4ccccc4c(-c4ccccc4)c4ccccc34)c3c2oc2ccccc23)c1 ; Fc1ccc(-c2ccc(-c3cccc4ccccc34)c3ccccc23)cc1 ; Fc1cccc(-c2ccc(-c3ccc4ccccc4c3)c3ccccc23)c1]

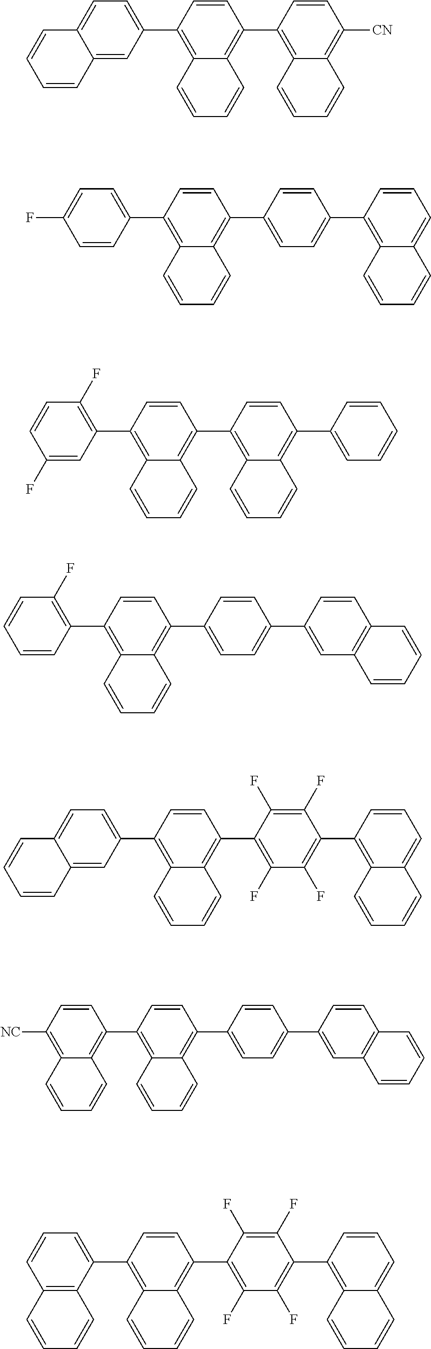

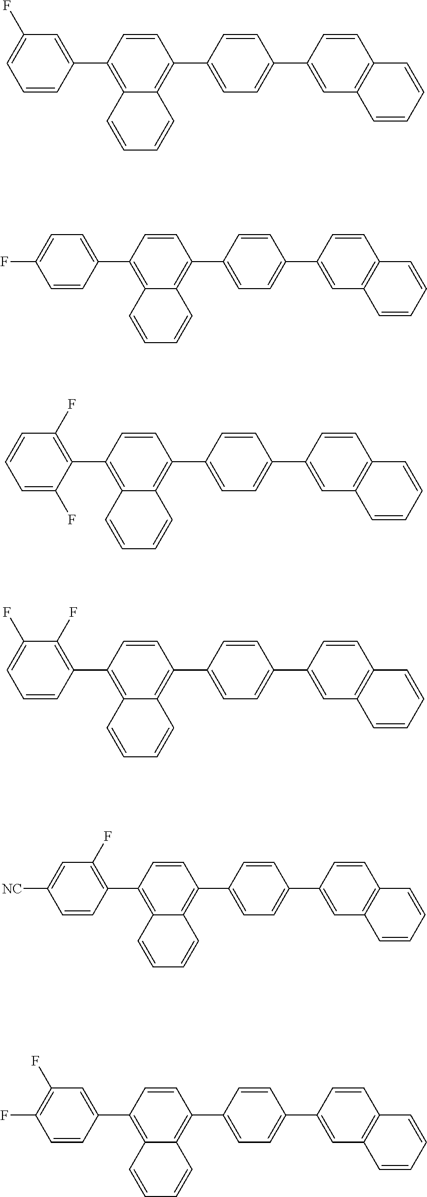 [SMILES: Fc1cccc(-c2ccc(-c3ccc(-c4ccc5ccccc5c4)cc3)c3ccccc23)c1 ; Fc1ccc(-c2ccc(-c3ccc(-c4ccc5ccccc5c4)cc3)c3ccccc23)cc1 ; Fc1cccc(F)c1-c1ccc(-c2ccc(-c3ccc4ccccc4c3)cc2)c2ccccc12 ; Fc1cccc(-c2ccc(-c3ccc(-c4ccc5ccccc5c4)cc3)c3ccccc23)c1F ; N#Cc1ccc(-c2ccc(-c3ccc(-c4ccc5ccccc5c4)cc3)c3ccccc23)c(F)c1 ; Fc1ccc(-c2ccc(-c3ccc(-c4ccc5ccccc5c4)cc3)c3ccccc23)cc1F]

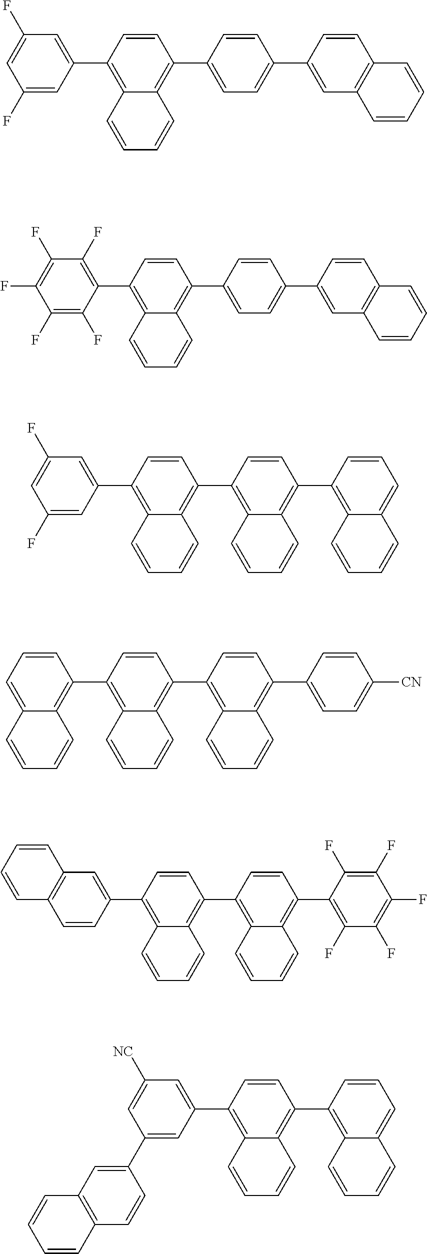 [SMILES: Fc1cc(F)cc(-c2ccc(-c3ccc(-c4ccc5ccccc5c4)cc3)c3ccccc23)c1 ; Fc1c(F)c(F)c(-c2ccc(-c3ccc(-c4ccc5ccccc5c4)cc3)c3ccccc23)c(F)c1F ; Fc1cc(F)cc(-c2ccc(-c3ccc(-c4cccc5ccccc45)c4ccccc34)c3ccccc23)c1 ; N#Cc1ccc(-c2ccc(-c3ccc(-c4cccc5ccccc45)c4ccccc34)c3ccccc23)cc1 ; Fc1c(F)c(F)c(-c2ccc(-c3ccc(-c4ccc5ccccc5c4)c4ccccc34)c3ccccc23)c(F)c1F ; N#Cc1cc(-c2ccc3ccccc3c2)cc(-c2ccc(-c3cccc4ccccc34)c3ccccc23)c1]

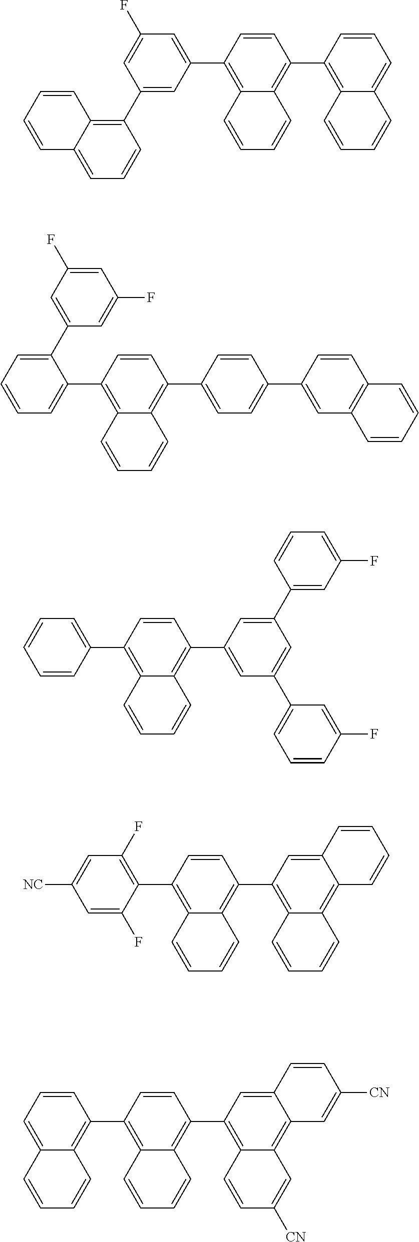 [SMILES: Fc1cc(-c2cccc3ccccc23)cc(-c2ccc(-c3cccc4ccccc34)c3ccccc23)c1 ; Fc1cc(F)cc(-c2ccccc2-c2ccc(-c3ccc(-c4ccc5ccccc5c4)cc3)c3ccccc23)c1 ; Fc1cccc(-c2cc(-c3cccc(F)c3)cc(-c3ccc(-c4ccccc4)c4ccccc34)c2)c1 ; N#Cc1cc(F)c(-c2ccc(-c3cc4ccccc4c4ccccc34)c3ccccc23)c(F)c1 ; N#Cc1ccc2cc(-c3ccc(-c4cccc5ccccc45)c4ccccc34)c3ccc(C#N)cc3c2c1]

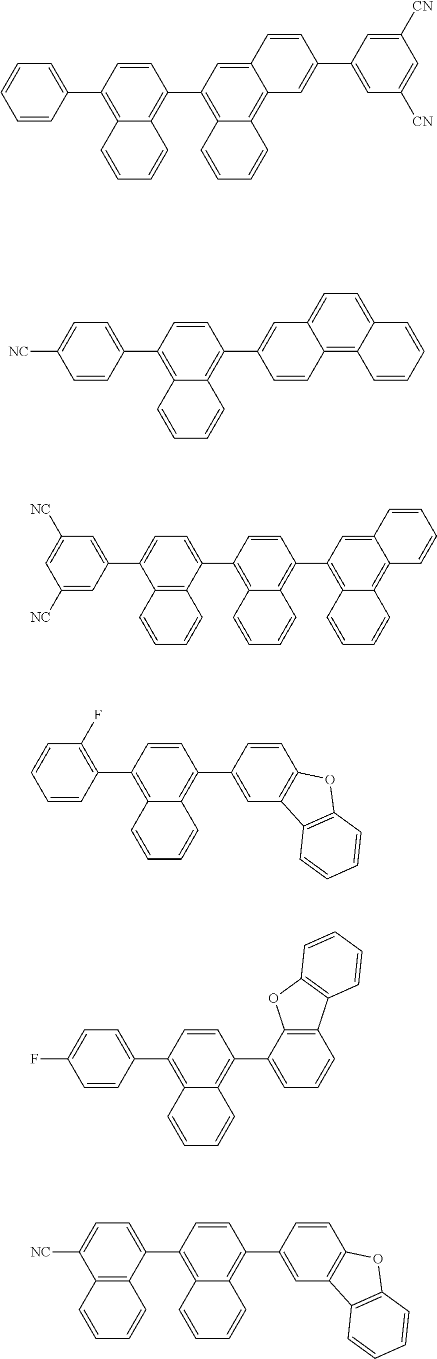 [SMILES: N#Cc1cc(C#N)cc(-c2ccc3cc(-c4ccc(-c5ccccc5)c5ccccc45)c4ccccc4c3c2)c1 ; N#Cc1ccc(-c2ccc(-c3ccc4c(ccc5ccccc54)c3)c3ccccc23)cc1 ; N#Cc1cc(C#N)cc(-c2ccc(-c3ccc(-c4cc5ccccc5c5ccccc45)c4ccccc34)c3ccccc23)c1 ; Fc1ccccc1-c1ccc(-c2ccc3oc4ccccc4c3c2)c2ccccc12 ; Fc1ccc(-c2ccc(-c3cccc4c3oc3ccccc34)c3ccccc23)cc1 ; N#Cc1ccc(-c2ccc(-c3ccc4oc5ccccc5c4c3)c3ccccc23)c2ccccc12]

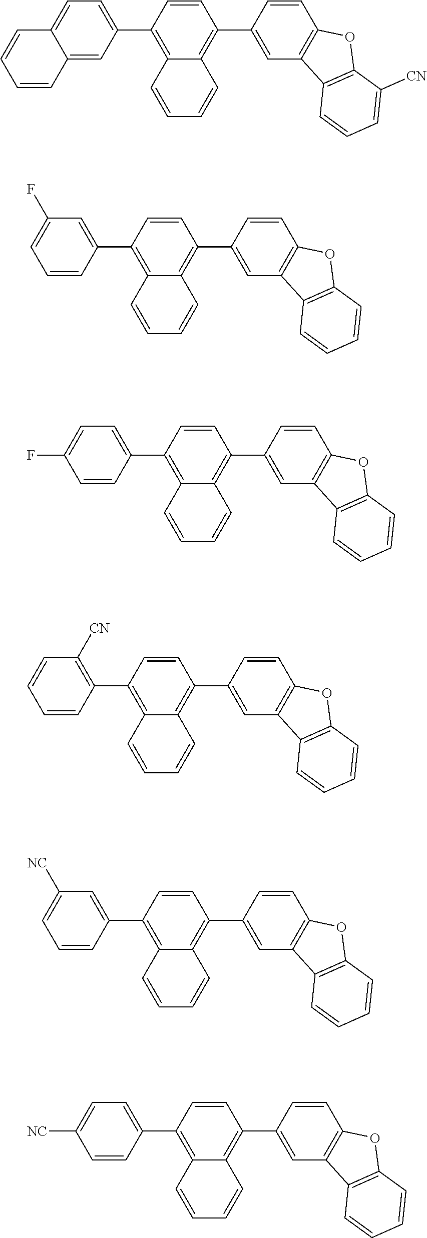 [SMILES: N#Cc1cccc2c1oc1ccc(-c3ccc(-c4ccc5ccccc5c4)c4ccccc34)cc12 ; Fc1cccc(-c2ccc(-c3ccc4oc5ccccc5c4c3)c3ccccc23)c1 ; Fc1ccc(-c2ccc(-c3ccc4oc5ccccc5c4c3)c3ccccc23)cc1 ; N#Cc1ccccc1-c1ccc(-c2ccc3oc4ccccc4c3c2)c2ccccc12 ; N#Cc1cccc(-c2ccc(-c3ccc4oc5ccccc5c4c3)c3ccccc23)c1 ; N#Cc1ccc(-c2ccc(-c3ccc4oc5ccccc5c4c3)c3ccccc23)cc1]

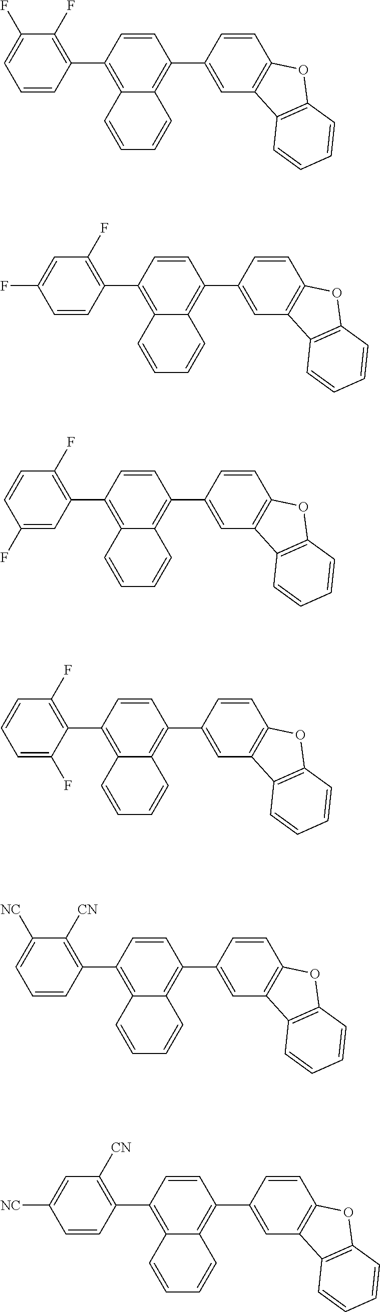 [SMILES: Fc1cccc(-c2ccc(-c3ccc4oc5ccccc5c4c3)c3ccccc23)c1F ; Fc1ccc(-c2ccc(-c3ccc4oc5ccccc5c4c3)c3ccccc23)c(F)c1 ; Fc1ccc(F)c(-c2ccc(-c3ccc4oc5ccccc5c4c3)c3ccccc23)c1 ; Fc1cccc(F)c1-c1ccc(-c2ccc3oc4ccccc4c3c2)c2ccccc12 ; N#Cc1cccc(-c2ccc(-c3ccc4oc5ccccc5c4c3)c3ccccc23)c1C#N ; N#Cc1ccc(-c2ccc(-c3ccc4oc5ccccc5c4c3)c3ccccc23)c(C#N)c1]

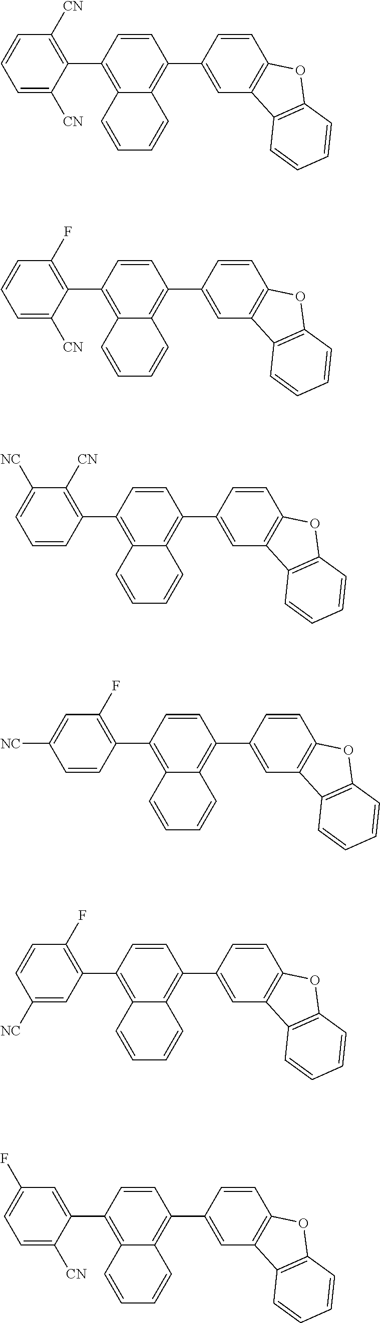 [SMILES: N#Cc1cccc(C#N)c1-c1ccc(-c2ccc3oc4ccccc4c3c2)c2ccccc12 ; N#Cc1cccc(F)c1-c1ccc(-c2ccc3oc4ccccc4c3c2)c2ccccc12 ; N#Cc1cccc(-c2ccc(-c3ccc4oc5ccccc5c4c3)c3ccccc23)c1C#N ; N#Cc1ccc(-c2ccc(-c3ccc4oc5ccccc5c4c3)c3ccccc23)c(F)c1 ; N#Cc1ccc(F)c(-c2ccc(-c3ccc4oc5ccccc5c4c3)c3ccccc23)c1 ; N#Cc1ccc(F)cc1-c1ccc(-c2ccc3oc4ccccc4c3c2)c2ccccc12]

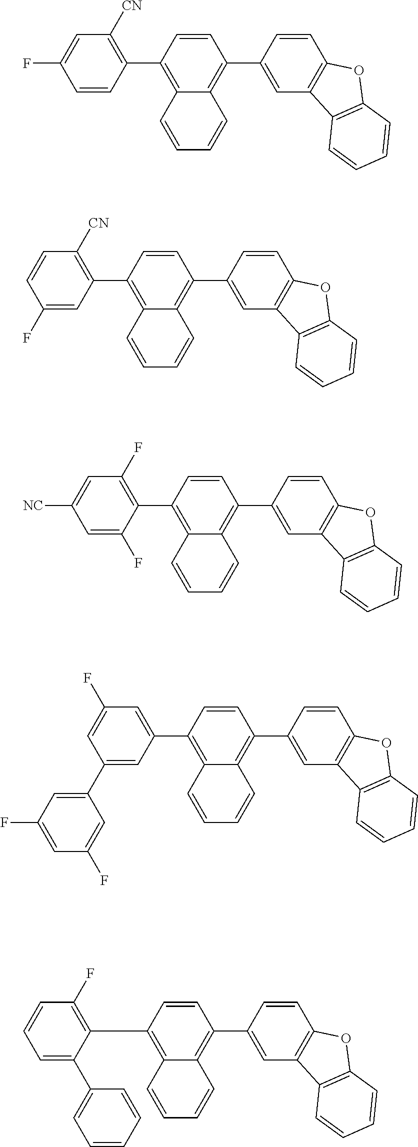 [SMILES: N#Cc1cc(F)ccc1-c1ccc(-c2ccc3oc4ccccc4c3c2)c2ccccc12 ; N#Cc1ccc(F)cc1-c1ccc(-c2ccc3oc4ccccc4c3c2)c2ccccc12 ; N#Cc1cc(F)c(-c2ccc(-c3ccc4oc5ccccc5c4c3)c3ccccc23)c(F)c1 ; Fc1cc(F)cc(-c2cc(F)cc(-c3ccc(-c4ccc5oc6ccccc6c5c4)c4ccccc34)c2)c1 ; Fc1cccc(-c2ccccc2)c1-c1ccc(-c2ccc3oc4ccccc4c3c2)c2ccccc12]

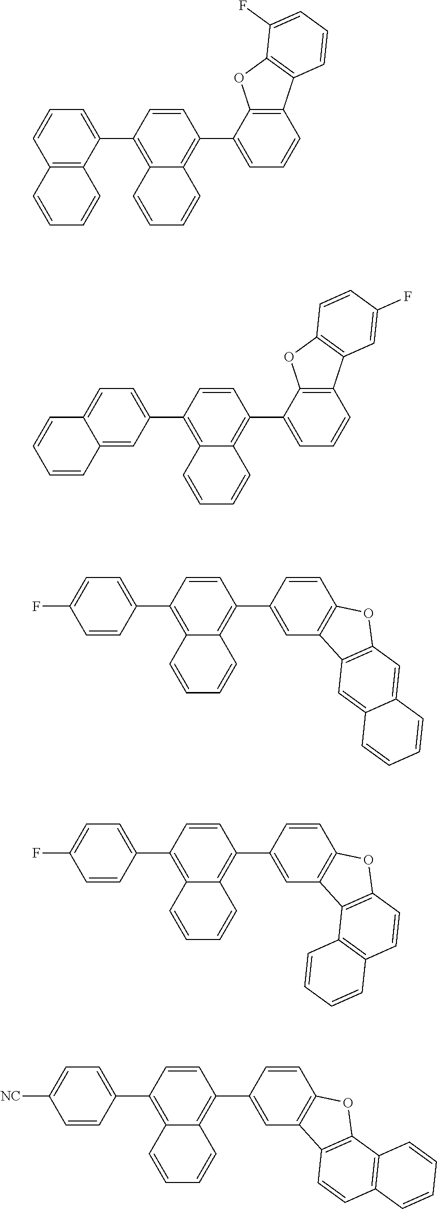 [SMILES: Fc1cccc2c1oc1c(-c3ccc(-c4cccc5ccccc45)c4ccccc34)cccc12 ; Fc1ccc2oc3c(-c4ccc(-c5ccc6ccccc6c5)c5ccccc45)cccc3c2c1 ; Fc1ccc(-c2ccc(-c3ccc4oc5cc6ccccc6cc5c4c3)c3ccccc23)cc1 ; Fc1ccc(-c2ccc(-c3ccc4oc5ccc6ccccc6c5c4c3)c3ccccc23)cc1 ; N#Cc1ccc(-c2ccc(-c3ccc4oc5c6ccccc6ccc5c4c3)c3ccccc23)cc1]

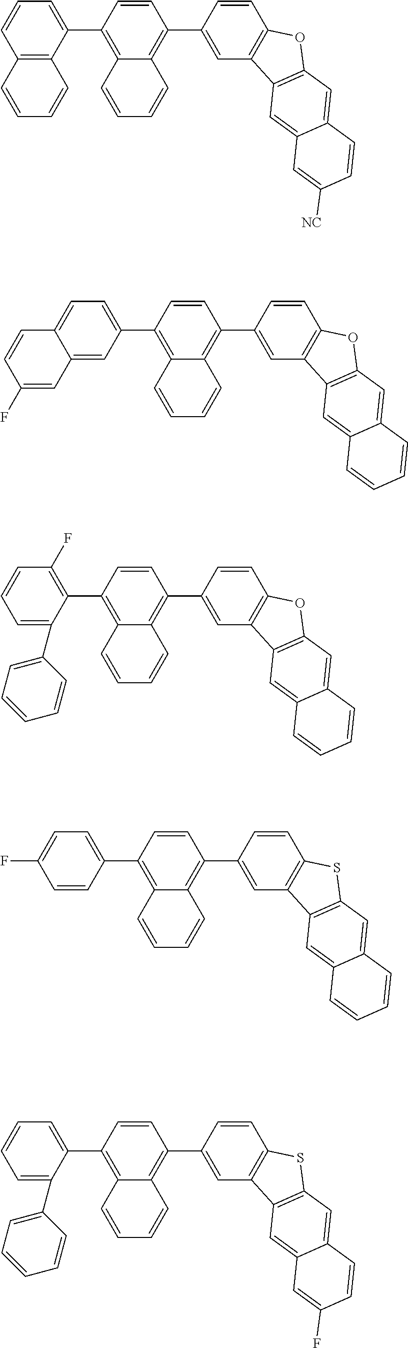 [SMILES: N#Cc1ccc2cc3oc4ccc(-c5ccc(-c6cccc7ccccc67)c6ccccc56)cc4c3cc2c1 ; Fc1ccc2ccc(-c3ccc(-c4ccc5oc6cc7ccccc7cc6c5c4)c4ccccc34)cc2c1 ; Fc1cccc(-c2ccccc2)c1-c1ccc(-c2ccc3oc4cc5ccccc5cc4c3c2)c2ccccc12 ; Fc1ccc(-c2ccc(-c3ccc4sc5cc6ccccc6cc5c4c3)c3ccccc23)cc1 ; Fc1ccc2cc3sc4ccc(-c5ccc(-c6ccccc6-c6ccccc6)c6ccccc56)cc4c3cc2c1]

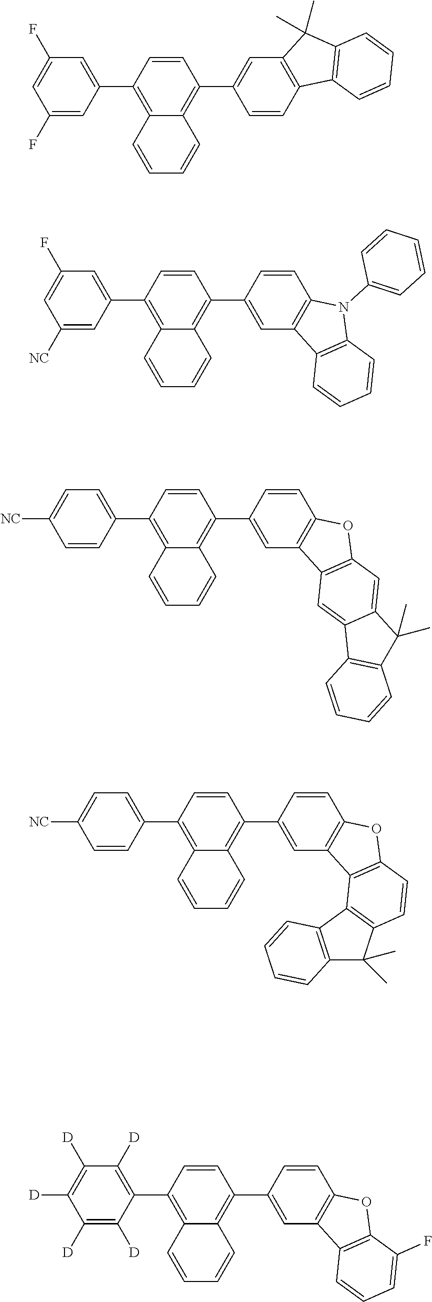 [SMILES: CC1(C)c2ccccc2-c2ccc(-c3ccc(-c4cc(F)cc(F)c4)c4ccccc34)cc21 ; N#Cc1cc(F)cc(-c2ccc(-c3ccc4c(c3)c3ccccc3n4-c3ccccc3)c3ccccc23)c1 ; CC1(C)c2ccccc2-c2cc3c(cc21)oc1ccc(-c2ccc(-c4ccc(C#N)cc4)c4ccccc24)cc13 ; CC1(C)c2ccccc2-c2c1ccc1oc3ccc(-c4ccc(-c5ccc(C#N)cc5)c5ccccc45)cc3c21 ; [2H]c1c([2H])c([2H])c(-c2ccc(-c3ccc4oc5c(F)cccc5c4c3)c3ccccc23)c([2H])c1[2H]]

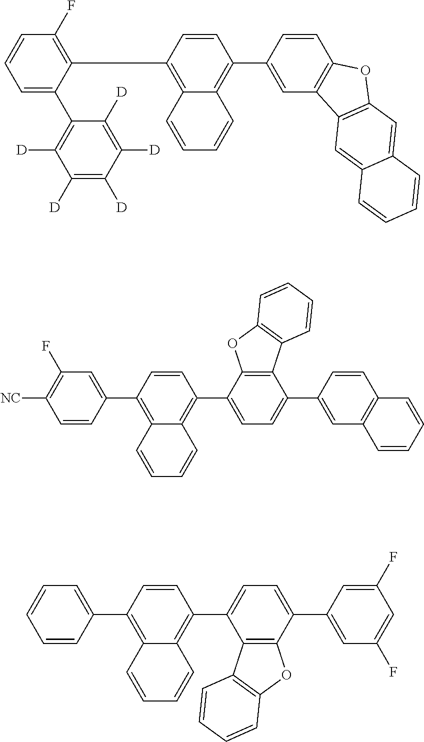 [SMILES: [2H]c1c([2H])c([2H])c(-c2cccc(F)c2-c2ccc(-c3ccc4oc5cc6ccccc6cc5c4c3)c3ccccc23)c([2H])c1[2H] ; N#Cc1ccc(-c2ccc(-c3ccc(-c4ccc5ccccc5c4)c4c3oc3ccccc34)c3ccccc23)cc1F ; Fc1cc(F)cc(-c2ccc(-c3ccc(-c4ccccc4)c4ccccc34)c3c2oc2ccccc23)c1]

In addition or alternatively, the organic mixture as described herein further comprises at least one other organic functional material, the at least one other organic functional material may be selected from the group consisting of a hole-injection material (HIM), a hole-transport material (HTM), a hole-blocking material (HBM), an electron-injection material (EIM), an electron-transport material (ETM), an electron-blocking material (EBM), an organic host material (Host), a singlet emitting material (fluorescent emitting material), a triplet emitting material (phosphorescent emitting material), a thermally activated delayed fluorescence material (TADF material), and an organic dye. These organic functional materials are described in details, for example, in WO2010135519A1, U.S.20090134784A1, and WO2011110277A1. The entire contents of the these three documents are incorporated herein for reference.

In some embodiments, E_{X} (i.e., min(|HOMO(H₁)-LUMO(H₂)|, |HOMO(H₂)-LUMO(H₁)|)) of the organic mixture is greater than or equal to S₁ of the at least one other organic functional material. A possible advantage of this is that the transition excited state of the organic mixture is able to rapidly transfer energy to the S₁ state of the at least one other organic functional material. Possible mechanisms of the energy transfer may be Foerster Transfer or Dexter Transfer.

In some embodiments, the organic mixture as described herein further comprises another fluorescent dopant material. The organic mixture as described herein can be used herein as host, and the content of the corresponding dopant should be ≤15 wt %, preferably ≤10 wt %, more preferably ≤8 wt %, further preferably ≤7 wt %, and most preferably ≤5 wt %. Further, the content of the corresponding dopant is preferably ≤4 wt %, more preferably ≤3 wt %, and most preferably ≤2 wt %.

In some embodiments, the organic mixture as described herein further comprises another TADF material.

The fluorescent dopant material (singlet emitter) and TADF material are described in details below (but not limited thereto).

### 1. Singlet Emitter

The singlet emitter tends to have a longer conjugate π-electron system. Hitherto, there have been many examples of styryl amines and derivatives thereof as disclosed in JP2913116B and WO2001021729A1, and indenofluorenes and derivatives thereof as disclosed in WO2008006449 and WO2007140847.

In some embodiments, the singlet emitter can be selected from the group consisting of monostyrylamines, distyrylamines, tristyrylamines, tetrastyrylamines, styrenphosphines, styrenethers, and arylamines.

A monostyrylamine refers to a compound, which comprises an unsubstituted or substituted styrenyl group and at least one amine, preferably an aromatic amine. A distyrylamine is meant a compound comprising two unsubstituted or substituted styrylgroups and at least one amine, preferably an aromatic amine. A ternarystyrylamine refers to a compound comprising three unsubstituted or substituted styryl groups and at least one amine, preferably an aromatic amine. A quaternarystyrylamine refers to a compound comprising four unsubstituted or substituted styryl groups and at least one amine, preferably an aromatic amine. A preferred styrene is stilbene, which may be further substituted. The corresponding phosphines and ethers are defined analogously as amines. Aryl amine or aromatic amine refers to a compound comprising three unsubstituted or substituted aromatic rings or heterocyclic systems directly linked to nitrogen. At least one of these aromatic or heterocyclic ring systems is preferably a fused ring system and preferably has at least 14 aromatic ring atoms. Preferred examples thereof include aromatic anthracene amine, aromatic anthracene diamine, aromatic pyrene amine, aromatic pyrene diamine, aromatic trolamine and aromatic trodiamine An aromatic anthraceneamine refers to a compound, where a diarylamine group is attached directly to the anthracene, preferably at the position 9. An aromatic anthracene diamine refers to a compound in which two diarylamine groups are directly attached to the anthracene, most preferably at positions 9, 10. Aromatic pyrene amines, aromatic pyrene diamines, aromatic dromamines, and aromatic dromenediamines are similarly defined, where the diarylamino group is most preferably attached to position 1 or 1,6 of pyrene.

Examples of singlet emitters based on vinylamine and arylamine are also preferred examples which may be found in the following patent documents: WO2006000388, WO2006058737, WO2006000389, WO2007065549, WO2007115610, U.S. Pat. No. 7,250,532B2, DE102005058557A1, CN1583691A, JP08053397A, U.S. Pat. No. 6,251,531B1, U.S.2006210830A, EP1957606A1, and U.S.20080113101A1, the whole contents of which are incorporated herein by reference.

Examples of singlet emitters based on distyrylbenzene and its derivatives may be found in U.S. Pat. No. 5,120,129.

Further preferred singlet emitter can be selected from the group consisting of indenofluorene-amine and indenofluorene-diamine, as disclosed in WO2006122630, benzoindenofluorene-amine and benzoindenofluorene-diamine, as disclosed in WO2008006449, dibenzoindenofluorene-amine and dibenzoindenofluorene-diamine, as disclosed in WO2007140847.

Other materials that can be used as singlet emitters include polycyclic aromatic hydrocarbon compounds, in particular selected from the derivatives of the following compounds: anthracene (such as 9,10-di(2-naphthyl)anthracene), naphthalene, tetraphenyl, phenanthrene, perylene (such as 2,5,8,11-tetra-t-butylatedylene, indenoperylene, phenylene (such as 4,4′-(bis (9-ethyl-3-carbazovinylene)-1,1′-biphenyl), periflanthene, decacyclene, coronene, fluorene, spirobifluorene, arylpyren (e.g., U.S.20060222886), arylenevinylene (e.g. U.S. Pat. Nos. 5,121,029, 5,130,603), cyclopentadiene such as tetraphenylcyclopentadiene, rubrene, coumarine, rhodamine, quinacridone, pyrane such as 4 (dicyanoethylene)-6-(4-dimethylaminostyryl-2-methyl)-4H-pyrane (DCM), thiapyran, bis (azinyl) imine-boron compounds (e.g. U.S.20070092753A1), bis (azinyl) methene compounds, carbostyryl compounds, oxazone, benzoxazole, benzothiazole, benzimidazole, and diketopyrrolopyrrole. Some singlet emitter materials may be found in the following patent documents: U.S.20070252517A1, U.S. Pat. Nos. 4,769,292, and 6,020,078. The patent documents listed above are specially incorporated herein by reference in their entirety.

Examples of some suitable singlet emitters are listed below:

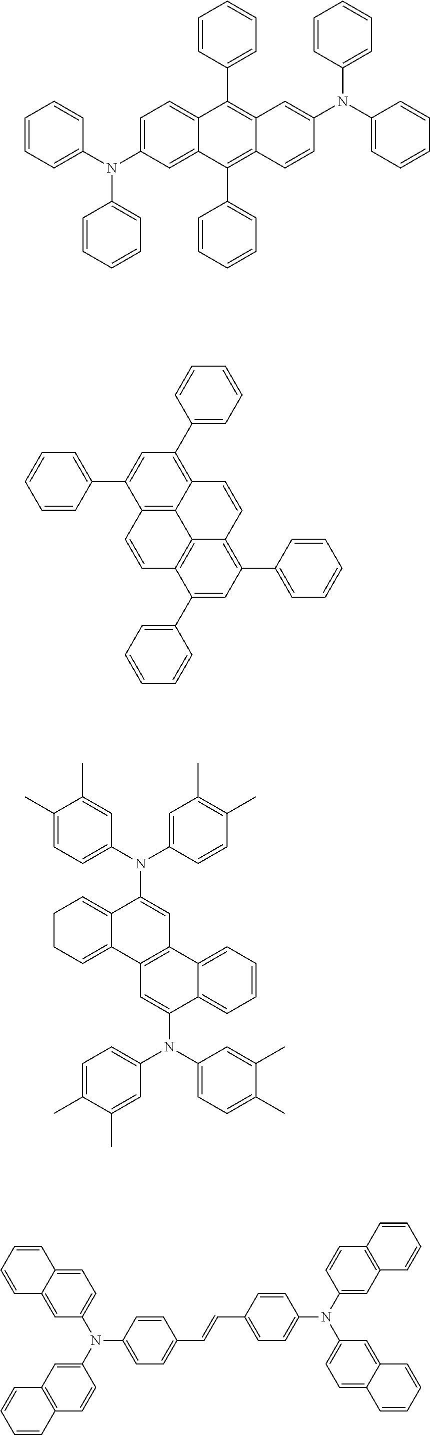

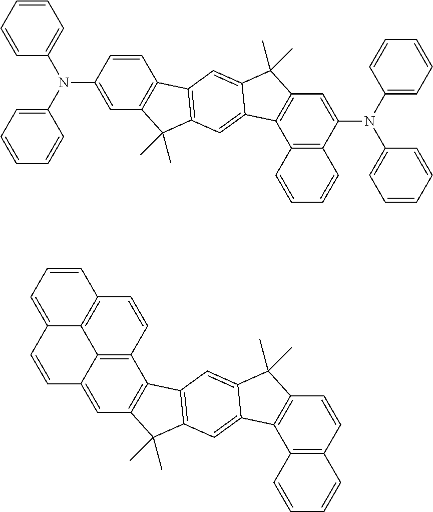

### 2. Thermally Activated Delayed Fluorescence Material (TADF material)

Traditional organic fluorescent materials can only emit light using 25% singlet excitonic luminescence formed by electrical excitation, and the devices have relatively low internal quantum efficiency (up to 25%). The phosphorescent material enhances the intersystem crossing due to the strong spin-orbit coupling of the heavy atom center, the singlet exciton and the triplet exciton luminescence formed by the electric excitation can be effectively utilized, so that the internal quantum efficiency of the device can reach 100%. However, the phosphorescent materials are expensive, the material stability is poor, and the device efficiency roll-off is a serious problem, which limit its application in OLED. Thermally activated delayed fluorescent (TADF) materials are the third generation of organic emitting materials developed after organic fluorescent materials and organic phosphorescent materials. This type of material generally has a small singlet-triplet energy level difference (ΔEst), and triplet excitons can be converted to singlet excitons by intersystem crossing. This can make full use of the singlet excitons and triplet excitons formed under electric excitation. The device can achieve 100% internal quantum efficiency. Meanwhile, due to the controllable structure, stable property, low cost, TADF materials without precious metals have a wide application prospect in the OLED field.

The TADF material needs to have a small singlet-triplet energy level difference, preferably ΔEst<0.3 eV, more preferably ΔEst<0.2 eV, and most preferably ΔEst<0.1 eV. In some embodiments, the TADF material has a relatively small ΔEst. In some embodiments, the TADF has a high fluorescence quantum efficiency. Some TADF materials may be found in the following patent documents: CN103483332A, TW201309696A, TW201309778A, TW201343874A, TW201350558A, U.S.20120217869A1, WO2013133359A1, WO2013154064A1, Adachi, et. al. Adv. Mater., 21, 2009, 4802, Adachi, et. al. Appl. Phys. Lett., 98, 2011, 083302, Adachi, et. al. Appl. Phys. Lett., 101, 2012, 093306, Adachi, et. al. Chem. Commun., 48, 2012, 11392, Adachi, et. al. Nature Photonics, 6, 2012, 253, Adachi, et. al. Nature, 492, 2012, 234, Adachi, et. al. J. Am. Chem. Soc, 134, 2012, 14706, Adachi, et. al. Angew. Chem. Int. Ed, 51, 2012, 11311, Adachi, et. al. Chem. Commun., 48, 2012, 9580, Adachi, et. al. Chem. Commun., 48, 2013, 10385, Adachi, et. al. Adv. Mater., 25, 2013, 3319, Adachi, et. al. Adv. Mater., 25, 2013, 3707, Adachi, et. al. Chem. Mater., 25, 2013, 3038, Adachi, et. al. Chem. Mater., 25, 2013,3766, Adachi, et. al. J. Mater. Chem. C., 1, 2013, 4599, Adachi, et. al. J. Phys. Chem. A., 117, 2013, 5607. The entire contents of the above listed patents or literature documents are hereby incorporated by reference.

Examples of some suitable TADF materials are listed below:

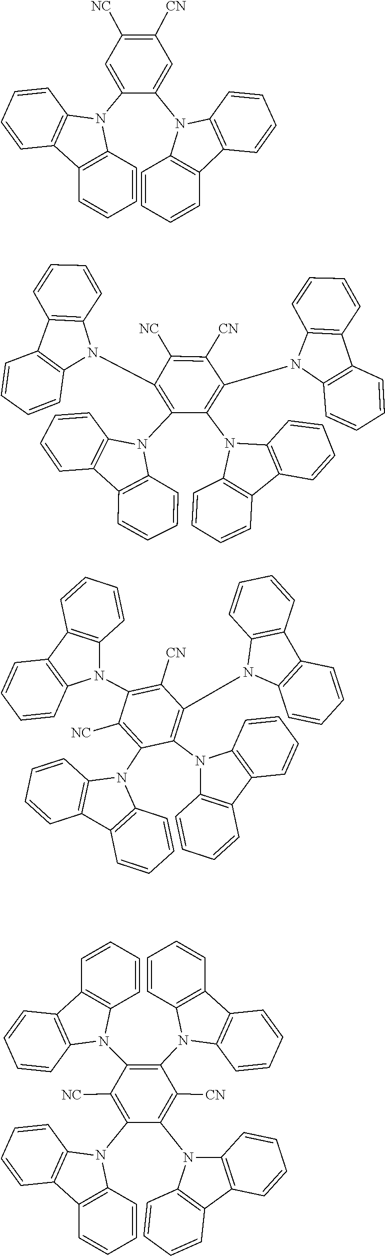

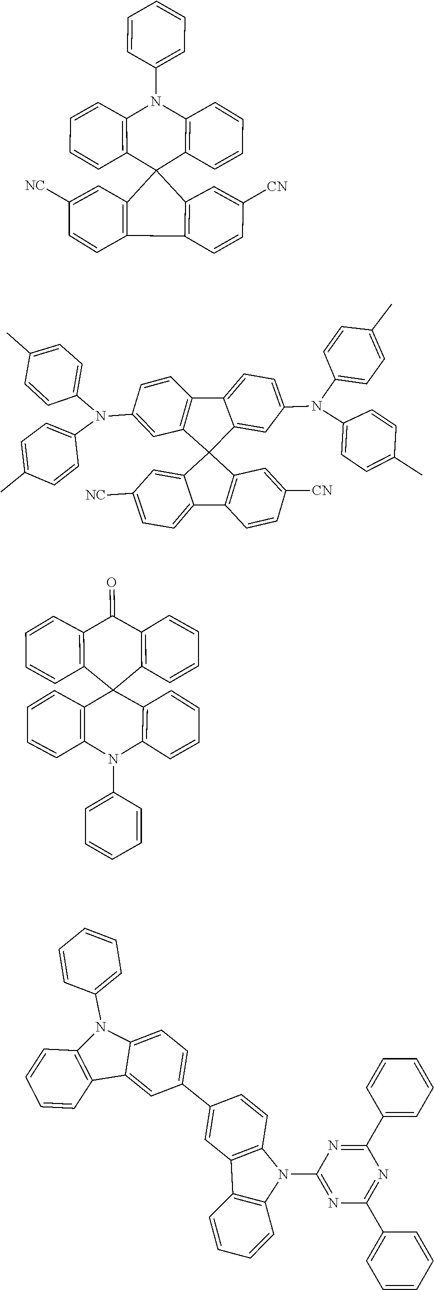

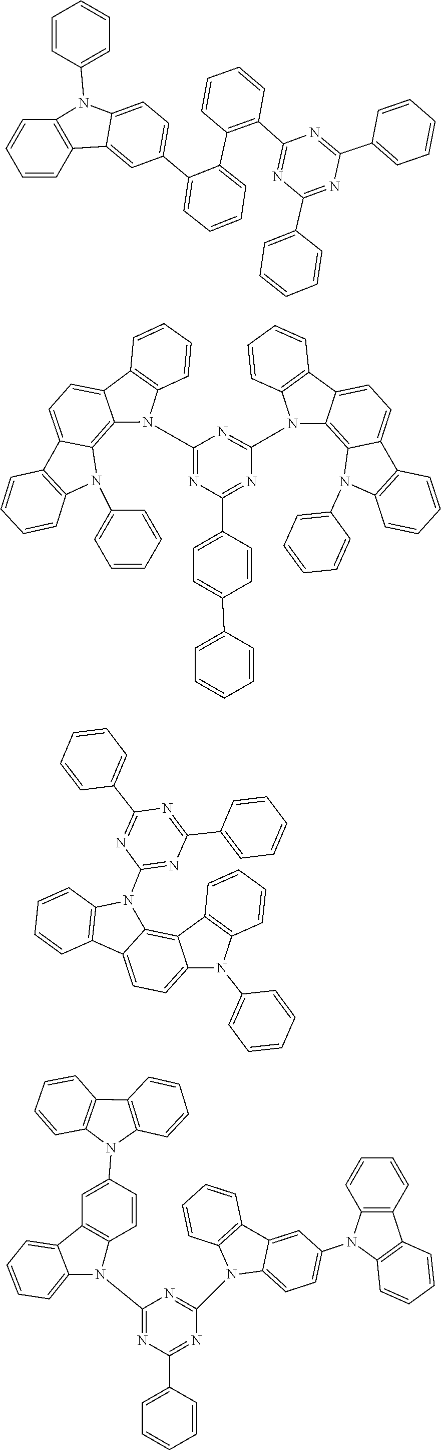

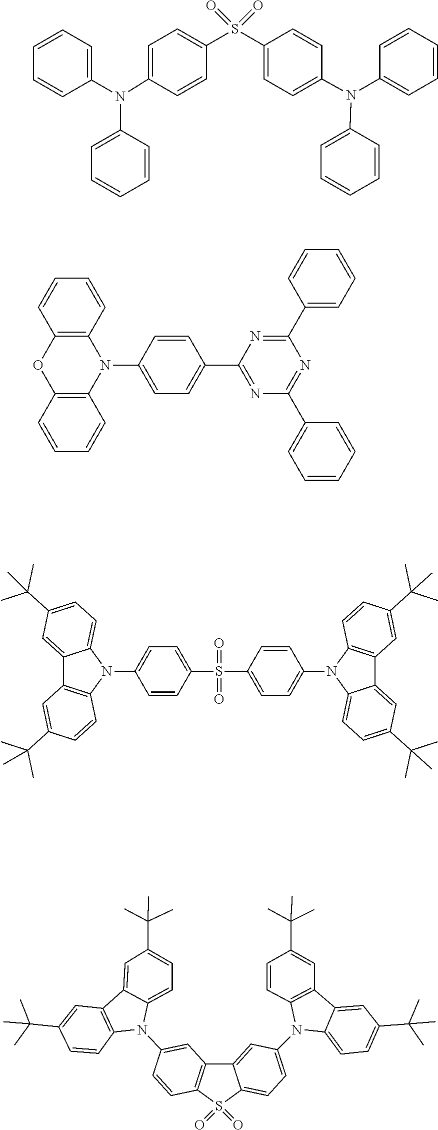

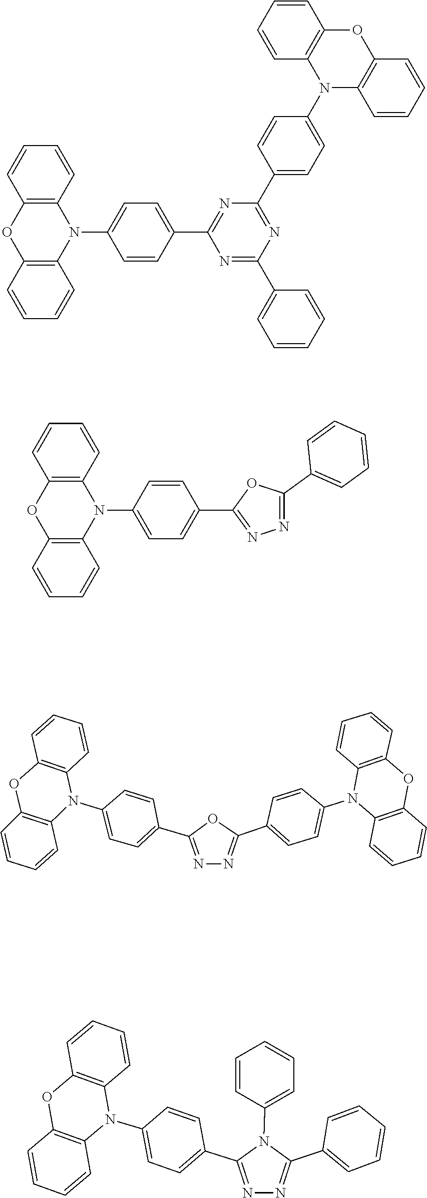 [SMILES: c1ccc(-c2nc(-c3ccc(N4c5ccccc5Oc5ccccc54)cc3)nc(-c3ccc(N4c5ccccc5Oc5ccccc54)cc3)n2)cc1 ; c1ccc(-c2nnc(-c3ccc(N4c5ccccc5Oc5ccccc54)cc3)o2)cc1 ; c1ccc2c(c1)Oc1ccccc1N2c1ccc(-c2nnc(-c3ccc(N4c5ccccc5Oc5ccccc54)cc3)o2)cc1 ; c1ccc(-c2nnc(-c3ccc(N4c5ccccc5Oc5ccccc54)cc3)n2-c2ccccc2)cc1]

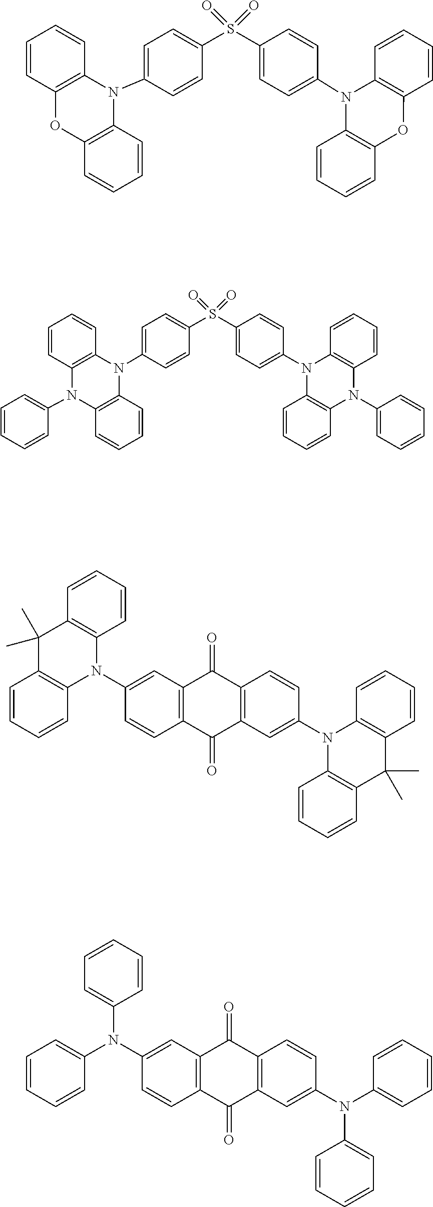

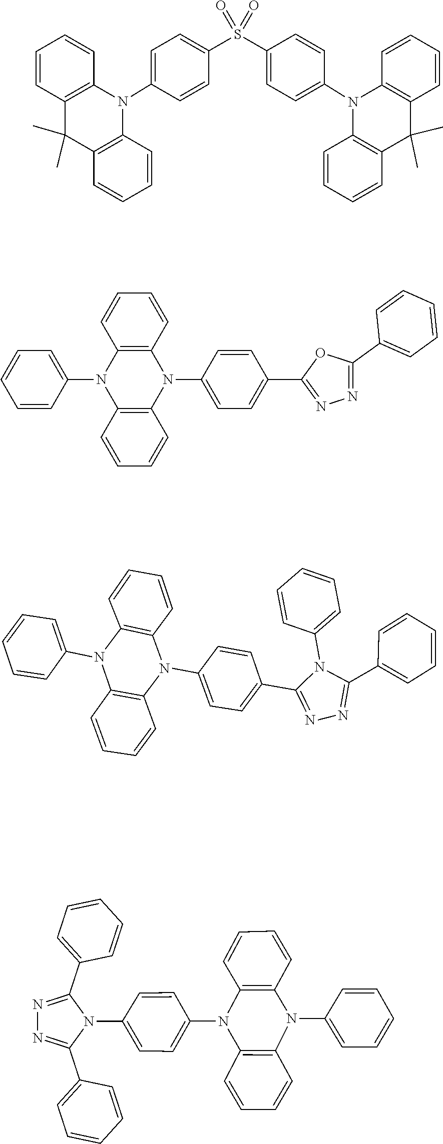

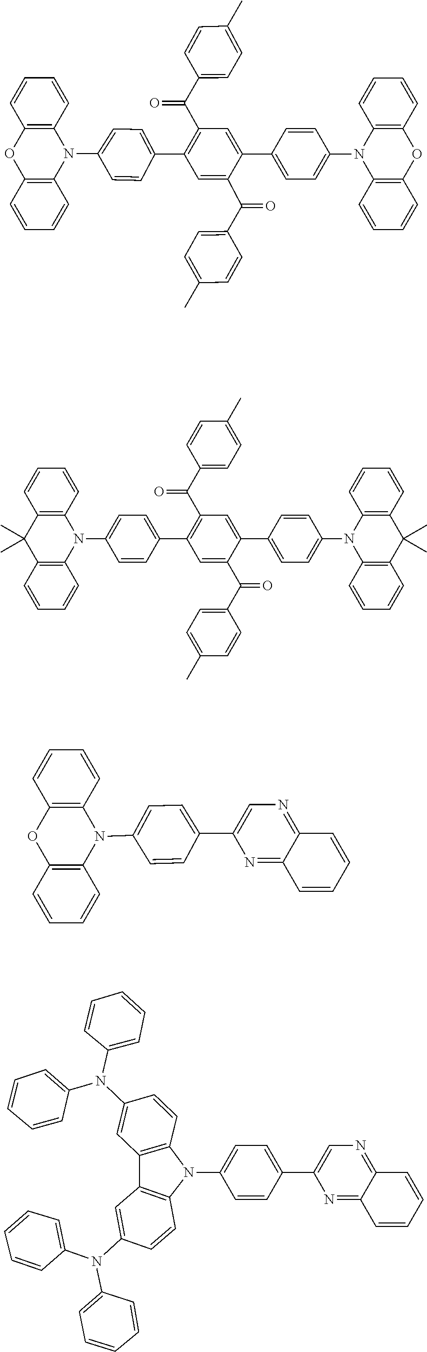

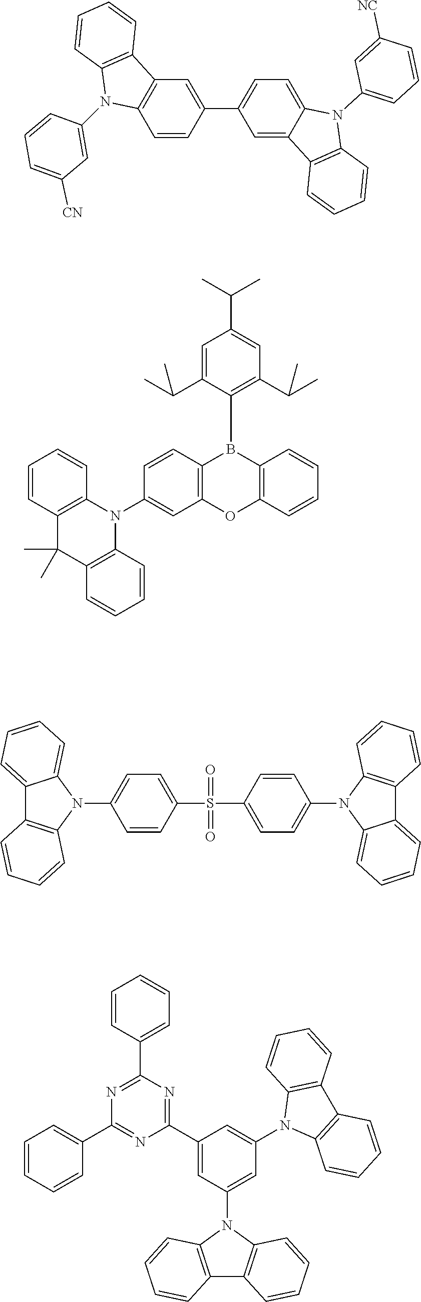 [SMILES: N#Cc1cccc(-n2c3ccccc3c3cc(-c4ccc5c(c4)c4ccccc4n5-c4cccc(C#N)c4)ccc32)c1 ; CC(C)c1cc(C(C)C)c(B2c3ccccc3Oc3cc(N4c5ccccc5C(C)(C)c5ccccc54)ccc32)c(C(C)C)c1 ; O=S(=O)(c1ccc(-n2c3ccccc3c3ccccc32)cc1)c1ccc(-n2c3ccccc3c3ccccc32)cc1 ; c1ccc(-c2nc(-c3ccccc3)nc(-c3cc(-n4c5ccccc5c5ccccc54)cc(-n4c5ccccc5c5ccccc54)c3)n2)cc1]

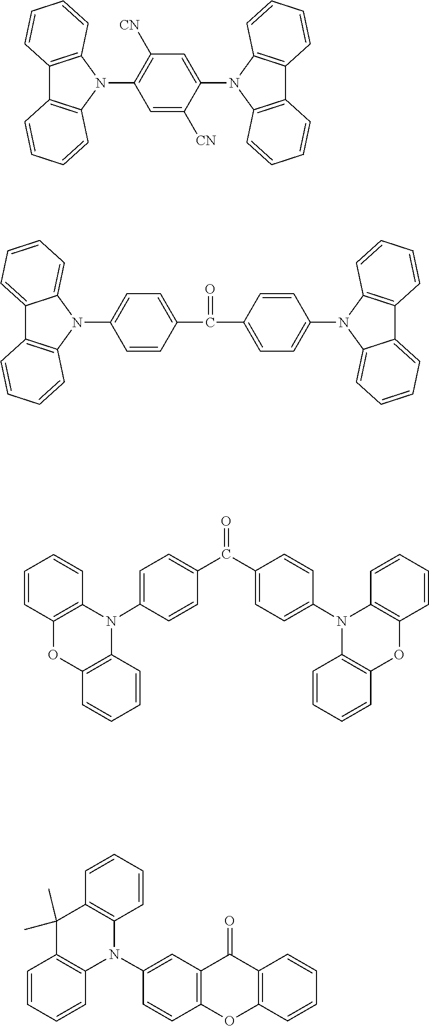

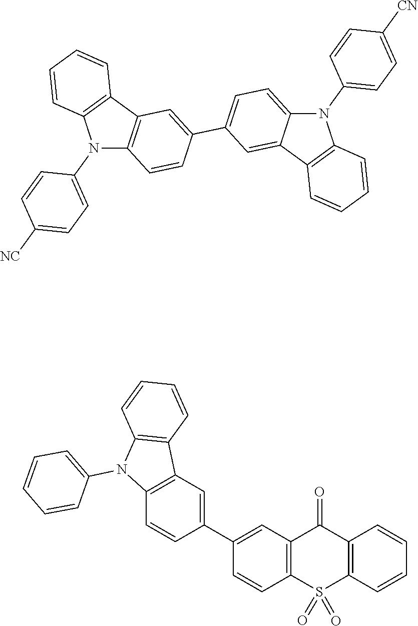 [SMILES: N#Cc1ccc(-n2c3ccccc3c3cc(-c4ccc5c(c4)c4ccccc4n5-c4ccc(C#N)cc4)ccc32)cc1 ; O=C1c2ccccc2S(=O)(=O)c2ccc(-c3ccc4c(c3)c3ccccc3n4-c3ccccc3)cc21]

The publications of organic functional material presented above are incorporated herein by reference for the purpose of disclosure.

The present disclosure also provides a material or an ink for printing electronic devices.

In some embodiments, the organic mixture as described herein has a solubility of ≥10 mg/ml in toluene at 25° C., preferably ≥15 mg/ml, and most preferably ≥20 mg/ml.

In another aspect, the present disclosure also provides a formulation comprising at least one organic mixture as described herein, and at least one organic solvent.

In some embodiments, the organic mixture of the formulation as described herein is used as a singlet host material.

In some embodiments, the formulation as described herein further comprises a dopant material.

In some embodiments, the formulation as described herein further comprises a thermally activated delayed fluorescence (TADF) material.

In some embodiments, the formulation as described herein further comprises a dopant material, and a TADF material.

In some embodiments, the formulation as described herein comprises a hole-transport material (HTM) and an organic mixture as described herein, more preferably, the HTM comprises a cross-linkable group.

In some embodiments, the formulation as described herein is a solution.

In some embodiments, the formulation as described herein is a dispersion.

The formulations in the embodiments of the present disclosure may comprise the organic mixture of 0.01 wt % to 20 wt %, preferably 0.1 wt % to 15 wt %, more preferably 0.2 wt % to 10 wt %, and most preferably 0.25 wt % to 5 wt %.

In some embodiments, the organic solvent of the formulation as described herein is selected from aromatics, heteroaromatics, esters, aromatic ketones, aromatic ethers, aliphatic ketones, aliphatic ethers, alicyclics, olefinic compounds, borate, phosphorate, or mixtures of two or more of them.

In some embodiments, the formulation as described herein comprising at least 50 wt % aromatic/heteroaromatic solvent, preferably at least 80 wt %, particularly preferably at least 90 wt %.

Examples of aromatic or heteroaromatic solvents as described herein include, but not limited to: 1-tetralone, 3-phenoxytoluene, acetophenone, 1-methoxynaphthalene, p-diisopropylbenzene, amylbenzene, tetrahydronaphthalene, cyclohexylbenzene, chloronaphthalene, 1,4-dimethylnaphthalene, 3-isopropylbiphenyl, p-methylisopropylbenzene, dipentylbenzene, o-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene, 1-methylnaphthalene, 1,2,4-trichlorobenzene, 1,3-dipropoxybenzene, 4,4-difluorodiphenylmethane, diphenyl ether, 1,2-dimethoxy-4-(1-propenyl) benzene, diphenylmethane, 2-phenylpyridine, 3-phenylpyridine, 2-phenoxymethyl ether, 2-phenoxytetrahydrofuran, ethyl-2-naphthyl ether, N-methyldiphenylamine, 4-isopropylbiphenyl, α, α-dichlorodiphenylmethane, 4-(3-phenylpropyl)pyridine, benzyl benzoate, 1,1-bis(3,4-dimethylphenyl) ethane, 2-isopropylnaphthalene, dibenzyl ether, etc.

In some embodiments, the suitable and preferred organic solvents include aliphatics, alicyclics, aromatics, amines, thiols, amides, nitriles, esters, ethers, polyethers, alcohols, diols, or polyols.

In some embodiments, the alcohol represents an organic solvent of the suitable class. The preferred alcohol includes alkylcyclohexanol, especially methylated aliphatic alcohol, naphthol, etc.

The organic solvent can be a cycloalkane, such as decahydronaphthalene.

The organic solvent can be used alone or as mixtures of two or more organic solvents.

In some embodiments, the formulation as described herein comprises an organic functional compound as described herein and at least one organic solvent, and further comprising another organic solvent. Examples of the another organic solvent include (but not limited to): methanol, ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 1,4 dioxane, acetone, methyl ethyl ketone, 1,2 dichloroethane, 3-phenoxytoluene, 1,1,1-trichloroethane, 1,1,2,2-tetrachloroethane, ethyl acetate, butyl acetate, dimethylformamide, dimethylacetamide, dimethylsulfoxide, tetrahydronaphthalene, decalin, indene, and/or mixtures thereof.

In some embodiments, the particularly suitable solvent for the present disclosure is a solvent having Hansen solubility parameters in the following ranges:
δ_{d} (dispersion force) is in the range of 17.0 to 23.2 MPa^{1/2}, especially in the range of 18.5 to 21.0 MPa^{1/2}.
δₚ (polarity force) is in the range of 0.2 to 12.5 MPa^{1/2}, especially in the range of 2.0 to 6.0 MPa^{1/2}.
δₕ (hydrogen bonding force) is in the range of 0.9 to 14.2 MPa^{1/2}, especially in the range of 2.0 to 6.0 MPa^{1/2}

In the formulation of the present disclosure, the boiling point parameter of the organic solvent should be taken into account when selecting the organic solvent. In the present disclosure, the boiling points of the organic solvents usually ≥150° C.; preferably ≥180° C.; more preferably ≥200° C.; further preferably ≥250° C.; and most preferably ≥275° C. or 300° C. The boiling points in these ranges are beneficial in terms for preventing nozzle clogging of the inkjet printhead. The organic solvent can be evaporated from the solution system to form a functional material film.

In some embodiments, the formulation of the present disclosure, where
1) the viscosity is in the range of 1 cps to 100 cps at 25° C.; and/or
2) the surface tension is in the range of 19 dyne/cm to 50 dyne/cm at 25° C.

In the formulation of the present disclosure, the surface tension parameter of the organic solvent should be taken into account when selecting the organic solvent. A suitable surface tension is required for the specific substrates and the specific printing methods. For example, for ink-jet printing, in some embodiments, the surface tension of the organic solvent at 25° C. is in the range of 19 dyne/cm to 50 dyne/cm, further in the range of 22 dyne/cm to 35 dyne/cm, and still further in the range of 25 dyne/cm to 33 dyne/cm.

In some embodiments, the surface tension of the ink as described herein at 25° C. is in the range of 19 dyne/cm to 50 dyne/cm; further in the range of 22 dyne/cm to 35 dyne/cm; and still further in the range of 25 dyne/cm to 33 dyne/cm.

In the formulation of the present disclosure, the viscosity parameters of the ink of the organic solvent should be taken into account when selecting the organic solvent. The viscosity can be adjusted by different methods, such as by the selection of suitable organic solvent and the concentration of functional materials in the ink. In some embodiments, the viscosity of the organic solvent is less than 100 cps, further less than 50 cps, and still further from 1.5 to 20 cps. The viscosity herein refers to the viscosity during printing at the ambient temperature that is generally at 15-30° C., further 18-28° C., still further 20-25° C., especially 23-25° C. The resulting formulation will be particularly suitable for ink-jet printing.

In some embodiments, the viscosity of the formulation as described herein at 25° C. is in the range of about 1 cps to 100 cps; particularly in the range of 1 cps to 50 cps; and most particularly in the range of 1.5 cps to 20 cps.

The ink obtained from the organic solvent satisfying the above-mentioned boiling point parameter, surface tension parameter and viscosity parameter can form a functional material film with uniform thickness and formulation property.

A further purpose of the present disclosure is to provide the use of the organic mixture and formulation thereof in organic electronic devices.

In addition or alternatively, the organic electronic device may be selected from an organic light emitting diode (OLED), an organic photovoltaic cell (OPV), an organic light emitting electrochemical cell (OLEEC), an organic field effect transistor (OFET), an organic light emitting field effect transistor, an organic laser, an organic spintronic electronic device, an organic sensor, or an organic plasmon emitting diode (OPED).

Another object of the present disclosure is to provide a method for producing the electronic device.

A specific technical solution comprises the following steps:

A functional layer is formed on a substrate by evaporating the organic mixture, or by co-evaporating with the at least one other organic functional material, or by printing or coating the formulation, where the printing or coating method may be selected from (but not limited to) ink- jet printing, nozzle printing, typographic printing, screen printing, dip coating, spin coating, blade coating, roller printing, torsion roll printing, planographic printing, flexographic printing, rotary printing, spray printing, brush coating, pad printing, slit die coating, etc.

The present disclosure further provides the use of the formulation as a printing ink in the preparation of organic electronic devices, particularly preferably by printing or coating processing methods.

Where suitable printing or coating techniques include, but not limited to ink-jet printing, typographic printing, screen printing, dip coating, spin coating, blade coating, roller printing, torsion roll printing, planographic printing, flexographic printing, rotary printing, spray coating, brush coating, pad printing, slit die coating, etc. Preferred techniques are gravure printing, screen printing, and ink-jet printing. Gravure printing and ink-jet printing will be applied in the embodiments of the present disclosure. The solution or dispersion may additionally comprise one or more components, such as surface active compounds, lubricants, wetting agents, dispersing agents, hydrophobic agents, binders, etc., which are used to adjust the viscosity and film forming properties, or to improve adhesion, etc. For more information on printing technologies and their requirements for solutions, such as solvent, concentration, and viscosity, etc., please refer to “Handbook of Print Media: Technologies and Production Methods”, edited by Helmut Kipphan, ISBN 3-540-67326-1.

The preparation methods as described herein, where the formed functional layer has a thickness of 5 nm to 1000 nm.

In yet another aspect, the present disclosure further provides an organic electronic device comprising an organic mixture as described herein, or a functional layer, which is prepared using the formulation as described herein. Generally, such organic electronic device comprises a cathode, an anode, and a functional layer disposed between the cathode and the anode, where the functional layer comprises an organic mixture as described herein.

In some embodiments, the organic electronic device as described herein is electroluminescent device, in particular an OLED, which comprises a substrate, an anode, at least one light emitting layer, and a cathode.

The substrate should be opaque or transparent. A transparent substrate could be used to produce a transparent light emitting device (for example: Bulovic et al. , Nature 1996, 380, p29, and Gu et al. , Appl. Phys. Lett. 1996, 68, p2606). The substrate can be rigid/flexible, e.g. it can be plastic, metal, semiconductor wafer, or glass. Preferably, the substrate has a smooth surface. Particularly desirable are substrates without surface defects. In some embodiments, the substrate is flexible and can be selected from a polymer film or plastic with a glass transition temperature Tg over 150° C., preferably over 200° C., more preferably over 250° C., and most preferably over 300° C. Examples of the suitable flexible substrate includes poly (ethylene terephthalate) (PET) and polyethylene glycol (2,6-naphthalene) (PEN).

The choice of anodes may include a conductive metal, a metal oxide, or a conductive polymer. The anode should be able to easily inject holes into a hole-injection layer (HIL), a hole-transport layer (HTL), or a light emitting layer. In some embodiments, the absolute value of the difference between the work function of the anode and the HOMO energy level of the emitter of the emitting layer, or the HOMO energy level/valence band energy level of the p-type semiconductor material for the hole-injection layer (HIL)/hole-transport layer (HTL)/electron-blocking layer (EBL) is less than 0.5 eV, preferably less than 0.3 eV, more preferably less than 0.2 eV. Examples of anode materials may include, but not limited to: Al, Cu, Au, Ag, Mg, Fe, Co, Ni, Mn, Pd, Pt, ITO, aluminum-doped zinc oxide (AZO), etc. Other suitable anode materials are known and can be readily selected for use by one of ordinary skill in the art. The anode material can be deposited using any suitable technique, such as a suitable physical vapor deposition method, including RF magnetron sputtering, vacuum thermal evaporation, e-beam, etc. In some embodiments, the anode is patterned. Patterned conductive ITO substrates are commercially available and can be used to produce the devices as present disclosure.

The choice of cathode may include a conductive metal and a metal oxide. The cathode should be able to easily inject electrons into the EIL, the ETL, or the directly into the emitting layer. In some embodiments, the absolute value of the difference between the work function of the cathode and the LUMO energy level of the emitter of the emitting layer, or the LUMO energy level/conduction band energy level of the n-type semiconductor material for electron-injection layer (EIL)/electron-transport layer (ETL)/hole-blocking layer (HBL) is less than 0.5 eV preferably less than 0.3 eV, most preferably less than 0.2 eV. In principle, all materials that may be used as cathodes for OLEDs are possible to apply as cathode materials for the present disclosure. Examples of cathode materials include, but not limited to: Al, Au, Ag, Ca, Ba, Mg, LiF/Al, MgAg alloys, BaF₂/Al, Cu, Fe, Co, Ni, Mn, Pd, Pt, ITO, etc. The cathode material can be deposited using any suitable technique, such as the suitable physical vapor deposition method, including RF magnetron sputtering, vacuum thermal evaporation, e-beam, etc.

The OLED device may also comprise other functional layers, such as a hole-injection layer (HIL), a hole-transport layer (HTL), an electron-blocking layer (EBL), an electron-injection layer (EIL), an electron-transport layer (ETL), and a hole-blocking layer (HBL). Materials suitable for use in these functional layers are described in details above and in WO2010135519A1, U.S.20090134784A1 and WO2011110277A1, the entire contents of the these three documents are hereby incorporated herein for reference.

In some embodiments, in the light emitting device as used herein, the light emitting layer is formed by vacuum evaporation deposition, and the evaporation source comprises an organic mixture of the present disclosure.

In some embodiments, the light emitting device of the present disclosure has a light-emitting layer prepared by printing the formulation of the present disclosure.

The electroluminescent device of the present disclosure has a light-emitting wavelength between 300 nm and 1000 nm, preferably between 350 nm and 900 nm, more preferably between 400 nm and 800 nm.

The present disclosure further provides the application of organic electronic devices in various electronic devices, including but not limited to display devices, lighting devices, light sources, sensors, etc.

The present disclosure further provides electronic devices comprising organic electronic devices of the present disclosure, including but not limited to display devices, lighting devices, light sources, sensors, etc.

The present disclosure will be described below in conjunction with the preferred embodiments, but the present disclosure is not limited to the following embodiments. It should be understood that the scope of the present disclosure is covered by the scope of the claims of the present disclosure, and those skilled in the art should understand that certain changes may be made to the embodiments of the present disclosure.

### Specific Embodiment

Synthesis Example 1: Synthesis of Compound H1-1

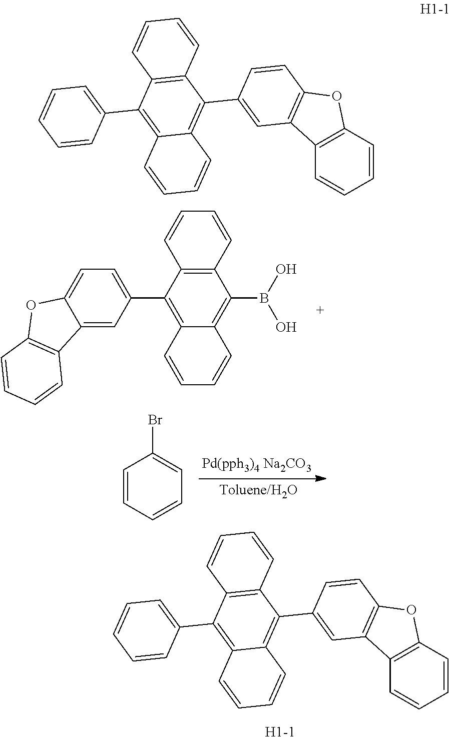

A solution of (10-(2-benzofuranyl)anthracene-9-boronic acid (7.76 g, 20 mmol), bromobenzene (3.1 g, 20 mmol) and 2.00 mol/l sodium carbonate (4.12 g, 40 mmol) was added into a three-necked flask, the resulting solution was dissolved in 100 ml of toluene by stirring under N₂ atmosphere protection, then Pd(pph₃)₄ (1.13 mg, 1 mmol) was added. After that, the resulting solution was stirred and reacted to reflux for 12 h. TLC and MS showed complete reaction, mainly for the target product, then the resulting solution was cooled. The sample was washed with 100 ml of saturated brine three times, dried over anhydrous sodium sulfate, then the solvent was removed by evaporation. After the reaction was terminated, the result was purified with DCM/PE (1:10) by column chromatography to yield (6.7 g, 80% yield) of a white solid (compound H1-1).

Synthesis Example 2-18: Synthesis of compounds H2-1 to H2-17.

Synthesis of compounds H2-1 to H2-17 was using the same synthetic technology route as compound H1-1, and were obtained in a one-step coupling reaction according to the intermediates shown below:

| Intermediate A | Intermediate B | Product | Yield |
|---|---|---|---|
| 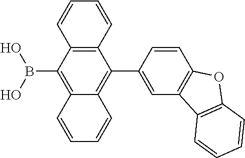 [SMILES: OB(O)c1c2ccccc2c(-c2ccc3oc4ccccc4c3c2)c2ccccc12] | 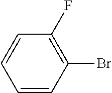 | 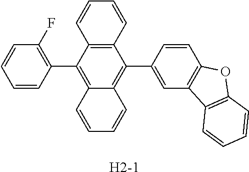 [SMILES: Fc1ccccc1-c1c2ccccc2c(-c2ccc3oc4ccccc4c3c2)c2ccccc12 ; "H2-1"] | 50% |
| 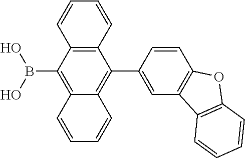 [SMILES: OB(O)c1c2ccccc2c(-c2ccc3oc4ccccc4c3c2)c2ccccc12] | 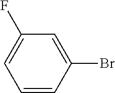 | 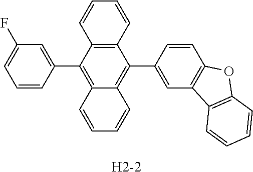 [SMILES: Fc1cccc(-c2c3ccccc3c(-c3ccc4oc5ccccc5c4c3)c3ccccc23)c1 ; "H2-2"] | 75% |
| 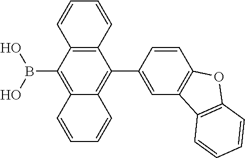 [SMILES: OB(O)c1c2ccccc2c(-c2ccc3oc4ccccc4c3c2)c2ccccc12] | 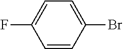 | 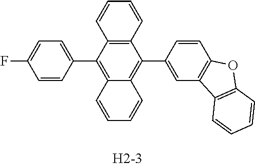 [SMILES: Fc1ccc(-c2c3ccccc3c(-c3ccc4oc5ccccc5c4c3)c3ccccc23)cc1] | 60% |
| 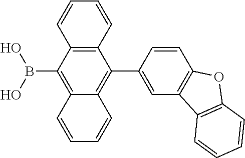 [SMILES: OB(O)c1c2ccccc2c(-c2ccc3oc4ccccc4c3c2)c2ccccc12] | 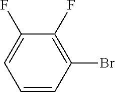 | 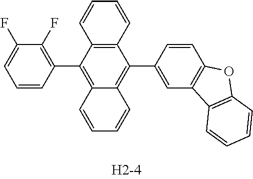 [SMILES: Fc1cccc(-c2c3ccccc3c(-c3ccc4oc5ccccc5c4c3)c3ccccc23)c1F] | 73% |
| 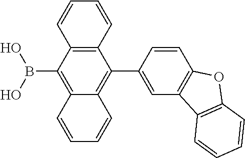 [SMILES: OB(O)c1c2ccccc2c(-c2ccc3oc4ccccc4c3c2)c2ccccc12] | 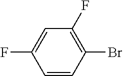 | 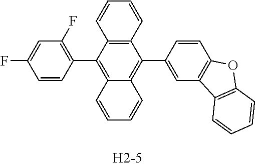 [SMILES: Fc1ccc(-c2c3ccccc3c(-c3ccc4oc5ccccc5c4c3)c3ccccc23)c(F)c1] | 75% |
| 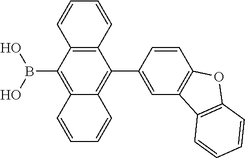 [SMILES: OB(O)c1c2ccccc2c(-c2ccc3oc4ccccc4c3c2)c2ccccc12] | 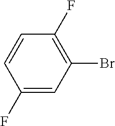 | 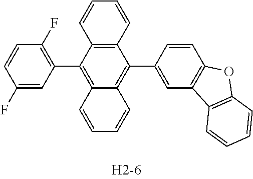 [SMILES: Fc1ccc(F)c(-c2c3ccccc3c(-c3ccc4oc5ccccc5c4c3)c3ccccc23)c1] | 65% |
| 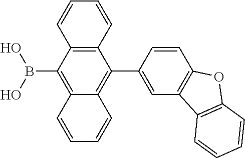 [SMILES: OB(O)c1c2ccccc2c(-c2ccc3oc4ccccc4c3c2)c2ccccc12] | 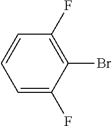 | 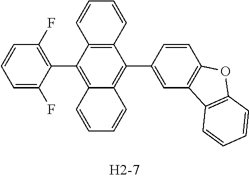 [SMILES: Fc1cccc(F)c1-c1c2ccccc2c(-c2ccc3oc4ccccc4c3c2)c2ccccc12] | 68% |
| 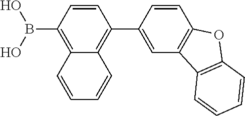 [SMILES: OB(O)c1ccc(-c2ccc3oc4ccccc4c3c2)c2ccccc12] | 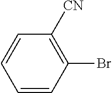 | 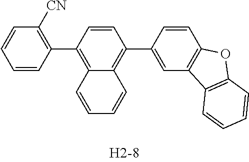 [SMILES: N#Cc1ccccc1-c1ccc(-c2ccc3oc4ccccc4c3c2)c2ccccc12] | 79% |
| 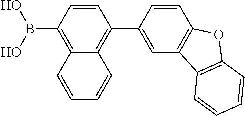 [SMILES: OB(O)c1ccc(-c2ccc3oc4ccccc4c3c2)c2ccccc12] | 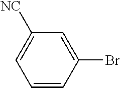 | 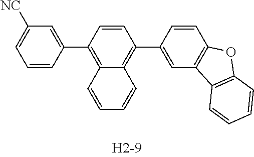 [SMILES: N#Cc1cccc(-c2ccc(-c3ccc4oc5ccccc5c4c3)c3ccccc23)c1] | 77% |
| 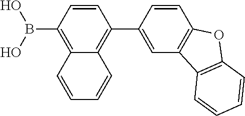 [SMILES: OB(O)c1ccc(-c2ccc3oc4ccccc4c3c2)c2ccccc12] | 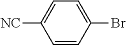 | 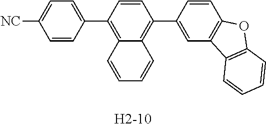 [SMILES: N#Cc1ccc(-c2ccc(-c3ccc4oc5ccccc5c4c3)c3ccccc23)cc1] | 56% |
| 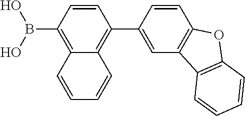 [SMILES: OB(O)c1ccc(-c2ccc3oc4ccccc4c3c2)c2ccccc12] | 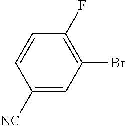 | 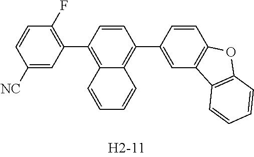 [SMILES: N#Cc1ccc(F)c(-c2ccc(-c3ccc4oc5ccccc5c4c3)c3ccccc23)c1] | 72% |
| 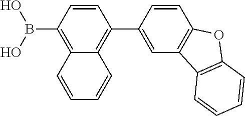 [SMILES: OB(O)c1ccc(-c2ccc3oc4ccccc4c3c2)c2ccccc12] | 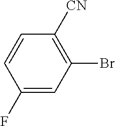 | 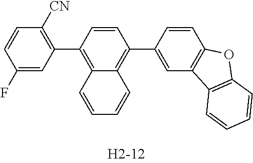 [SMILES: N#Cc1ccc(F)cc1-c1ccc(-c2ccc3oc4ccccc4c3c2)c2ccccc12] | 80% |
| 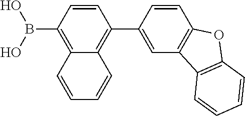 [SMILES: OB(O)c1ccc(-c2ccc3oc4ccccc4c3c2)c2ccccc12] | 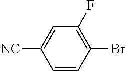 | 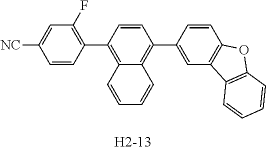 [SMILES: N#Cc1ccc(-c2ccc(-c3ccc4oc5ccccc5c4c3)c3ccccc23)c(F)c1] | 83% |
| 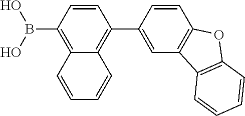 [SMILES: OB(O)c1ccc(-c2ccc3oc4ccccc4c3c2)c2ccccc12] | 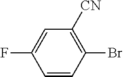 | 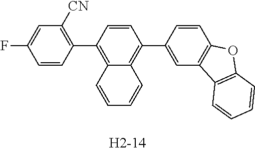 [SMILES: N#Cc1cc(F)ccc1-c1ccc(-c2ccc3oc4ccccc4c3c2)c2ccccc12] | 70% |
| 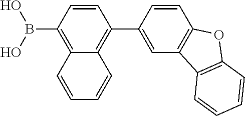 [SMILES: OB(O)c1ccc(-c2ccc3oc4ccccc4c3c2)c2ccccc12] | 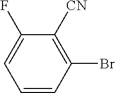 | 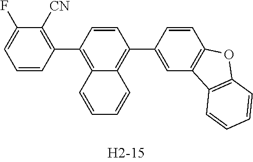 [SMILES: N#Cc1c(F)cccc1-c1ccc(-c2ccc3oc4ccccc4c3c2)c2ccccc12] | 74% |
| 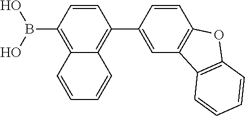 [SMILES: OB(O)c1ccc(-c2ccc3oc4ccccc4c3c2)c2ccccc12] | 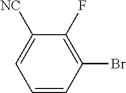 | 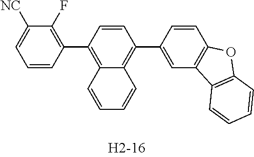 [SMILES: N#Cc1cccc(-c2ccc(-c3ccc4oc5ccccc5c4c3)c3ccccc23)c1F] | 65% |
| 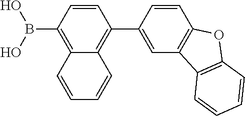 [SMILES: OB(O)c1ccc(-c2ccc3oc4ccccc4c3c2)c2ccccc12] | 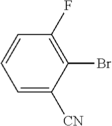 | 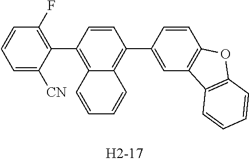 [SMILES: N#Cc1cccc(F)c1-c1ccc(-c2ccc3oc4ccccc4c3c2)c2ccccc12] | 69% |

The energy level of the organic material can be calculated by quantum computation, for example, using TD-DFT (time-dependent density functional theory) by Gaussian09W (Gaussian Inc.), the specific simulation methods of which can be found in WO2011141110. Firstly, the molecular geometry is optimized by semi-empirical method “Ground State/Semi-empirical/Default Spin/AM1” (Charge 0/Spin Singlet), and then the energy structure of organic molecules is calculated by TD-DFT (time-dependent density functional theory) “TD-SCF/DFT/Default Spin/B3PW91” and the basis set “6-31G (d)” (Charge 0/Spin Singlet). The HOMO and LUMO levels are calculated using the following calibration formula, where S1 and T1 are used directly.

HOMO(eV)=((HOMO(G)×27.212)−0.9899)/1.1206

LUMO(eV)=((LUMO(G)×27.212)−2.0041)/1.385

Where HOMO(G) and LUMO(G) are the direct calculation results of Gaussian 09W, in units of Hartree. The results are shown in Table 2 below:

TABLE 2

| | Homo | Lumo | Singlet | Triplet | | |
|---|---|---|---|---|---|---|
| | Corr. | Corr. | S1 | T1 | f | Molecular |
| Compound | [eV] | [eV] | [eV] | [eV] | (S1) | Mass |
| H1-1 | −5.56 | −2.70 | 3.18 | 1.68 | 0.1827 | 420.15 |
| H2-1 | −5.60 | −2.73 | 3.19 | 1.68 | 0.1828 | 438.14 |
| H2-2 | −5.63 | −2.76 | 3.19 | 1.68 | 0.1822 | 438.14 |
| H2-3 | −5.63 | −2.75 | 3.18 | 1.68 | 0.1828 | 438.14 |
| H2-4 | −5.67 | −2.79 | 3.19 | 1.68 | 0.1822 | 456.13 |
| H2-5 | −5.67 | −2.78 | 3.19 | 1.68 | 0.1829 | 456.13 |
| H2-6 | −5.67 | −2.79 | 3.19 | 1.68 | 0.1825 | 456.13 |
| H2-7 | −5.64 | −2.76 | 3.19 | 1.67 | 0.1822 | 456.13 |
| H2-8 | −6.07 | −2.63 | 3.59 | 2.51 | 0.239 | 395.13 |
| H2-9 | −6.15 | −2.62 | 3.61 | 2.51 | 0.1899 | 395.13 |
| H2-10 | −6.17 | −2.77 | 3.58 | 2.48 | 0.4619 | 395.13 |
| H2-11 | −6.14 | −2.68 | 3.58 | 2.50 | 0.3077 | 413.12 |
| H2-12 | −6.16 | −2.66 | 3.58 | 2.50 | 0.3198 | 413.12 |
| H2-13 | −6.19 | −2.84 | 3.56 | 2.48 | 0.3709 | 413.12 |
| H2-14 | −6.13 | −2.73 | 3.55 | 2.51 | 0.1649 | 413.12 |
| H2-15 | −6.13 | −2.74 | 3.57 | 2.50 | 0.2473 | 413.12 |
| H2-16 | −6.17 | −2.69 | 3.59 | 2.51 | 0.1231 | 413.12 |
| H2-17 | −6.11 | −2.70 | 3.55 | 2.53 | 0.1415 | 413.12 |
| D1 | −5.51 | −2.85 | 3.11 | 1.65 | 0.1958 | 456.19 |
| D2 | −5.55 | −2.88 | 3.11 | 1.65 | 0.2298 | 546.20 |
| D3 | −5.32 | −2.90 | 2.42 | 1.54 | 0.1335 | 512.23 |

Comparative Synthesis Example 1: Synthesis of Comparative Compound D1

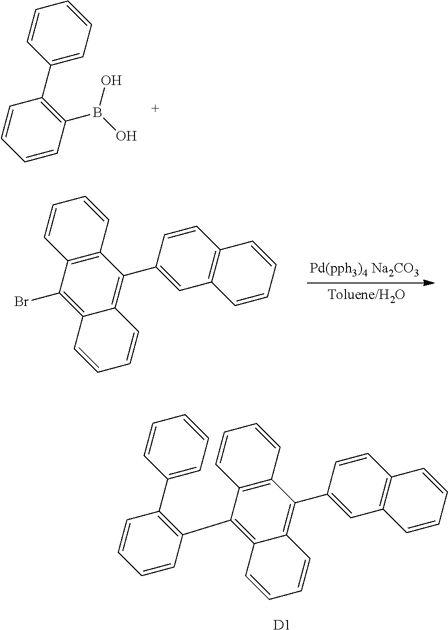

A solution of [1,1′-biphenyl]-2-boronic acid (3.96 g, 20 mmol), 9-bromo-10-(2-naphthalenyl)anthracene (7.66 g, 20 mmol) and 2.00 mol/l sodium carbonate (4.12 g, 40 mmol) was added into a three-necked flask the resulting solution was dissolved in 100 ml of toluene by stirring under N₂ atmosphere protection, then Pd(pph₃)₄ (1.13 mg, 1 mmol) was added. After that, the resulting solution was stirred and reacted to reflux for 12 h. TLC and MS showed complete reaction, mainly for the target product, then the resulting solution was cooled. The sample was washed with 100 ml of saturated brine three times, dried over anhydrous sodium sulfate, then the solvent was removed by evaporation. After the reaction was terminated, the result was purified with DCM/PE (1:10) by column chromatography to yield (7.12 g, 78% yield) of a white solid (compound D1).

Comparative Synthesis Example 2: Synthesis of Comparative Compound D2

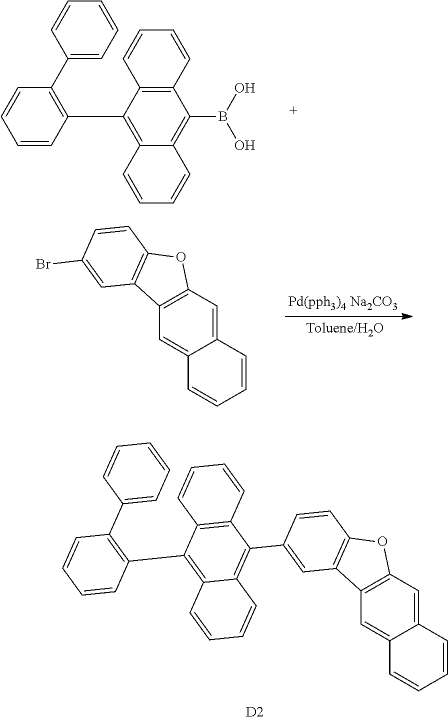

A solution of (10-([1,1′-biphenyl]-2-1)anthracene-9-boronic acid (7.48 g, 20 mmol), 2-bromobenzo[B]naphtho[2,3-D]furan (5.94 g, 20 mmol), and 2.00 mol/l sodium carbonate (4.12 g, 40 mmol) was added into a three-necked flask the resulting solution was dissolved in 100 ml of toluene by stirring under N₂ atmosphere protection, then Pd(pph₃)₄ (1.13 mg, 1 mmol) was added. After that, the resulting solution was stirred and reacted to reflux for 12 h. TLC and MS showed complete reaction, mainly for the target product, then the resulting solution was cooled. The sample was washed with 100 ml of saturated brine three times, dried over anhydrous sodium sulfate, then the solvent was removed by evaporation. After the reaction was terminated, the result was purified with DCM/PE (1:10) by column chromatography to yield (8.73 g, 80% yield) of a white solid (compound D2).

The example mixture was mixed in the following manner:

| Mixture | Compound H1 | Compound H2 | Ratio |
|---|---|---|---|
| Mixture 1 | H1-1 | H2-1 | 1:1 |
| Mixture 2 | H1-1 | H2-2 | 1:1 |
| Mixture 3 | H1-1 | H2-3 | 1:1 |
| Mixture 4 | H1-1 | H2-4 | 1:1 |
| Mixture 5 | H1-1 | H2-5 | 1:1 |
| Mixture 6 | H1-1 | H2-6 | 1:1 |
| Mixture 7 | H1-1 | H2-7 | 1:1 |
| Mixture 8 | H1-1 | H2-8 | 1:1 |
| Mixture 9 | H1-1 | H2-9 | 1:1 |
| Mixture 10 | H1-1 | H2-10 | 1:1 |
| Mixture 11 | H1-1 | H2-11 | 1:1 |
| Mixture 12 | H1-1 | H2-12 | 1:1 |
| Mixture 13 | H1-1 | H2-13 | 1:1 |
| Mixture 14 | H1-1 | H2-14 | 1:1 |
| Mixture 15 | H1-1 | H2-15 | 1:1 |
| Mixture 16 | H1-1 | H2-16 | 1:1 |
| Mixture 17 | H1-1 | H2-17 | 1:1 |

Two compounds in the mixture with equal equivalents were heated under vacuum until completely molten, mixed by stirring, and cooled to room temperature, and then grinded.

Preparation and characterization of OLEDs:

Materials used for various layer of OLEDs;

HIL: a triarylamine derivative;

HTL: a triarylamine derivative;

Host: mixture 1 to mixture 17, comparative compound D1 to comparative compound D2;

Dopant: an aromatic amine derivative K1.

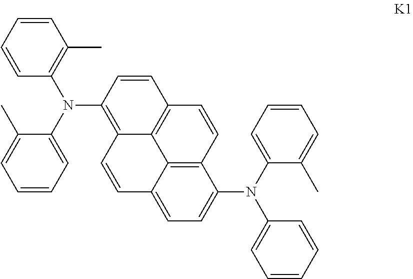 [SMILES: Cc1ccccc1N(c1ccccc1)c1ccc2ccc3c(N(c4ccccc4C)c4ccccc4C)ccc4ccc1c2c43]

The preparation steps of OLEDs with ITO/HIL (50 nm)/HTL (35 nm)/Host: 3% Dopant (25 nm)/ETL (28 nm)/LiQ (1 nm)/A1 (150 nm)/cathode are as follows:
a. Cleaning of the conductive glass substrate: prior to first-time use, the substrates are washed with various solvents (such as chloroform, ketone, or isopropyl alcohol), and then treated with UV and ozone;
b. HIL (50 nm), HTL (35 nm), EML (25 nm), and ETL (28 nm): thermal evaporation deposition in high vacuum (1×10⁻⁶ mbar);
c. Cathode: thermal evaporation deposition in high vacuum (1×10⁻⁶ mbar) with LiQ/Al (1 nm/150 nm);
d. Packaging: packaging the device in a nitrogen-regulated glove box with UV curable resin.

The current-voltage (J-V) characteristics of each OLED were studied. The current efficiency, device lifetime and the external quantum efficiency were summarized in Table 3. The experimental results of the efficiencies are shown in Table 3 below (Normalized by taking Example 2 as 100%):

TABLE 3

| Example | Host Material | Voltage (V) | Efficiency(cd/A) |
|---|---|---|---|
| Example 1 | Mixture 1 | 3.5 | 110% |
| Example 2 | Mixture 2 | 3.5 | 103% |
| Example 3 | Mixture 3 | 3.6 | 120% |
| Example 4 | Mixture 4 | 3.6 | 125% |
| Example 5 | Mixture 5 | 3.6 | 115% |
| Example 6 | Mixture 6 | 3.6 | 130% |
| Example 7 | Mixture 7 | 3.5 | 104% |
| Example 8 | Mixture 8 | 3.7 | 128% |
| Example 9 | Mixture 9 | 3.7 | 135% |
| Example 10 | Mixture 10 | 3.7 | 130% |
| Example 11 | Mixture 11 | 3.6 | 141% |
| Example 12 | Mixture 12 | 3.6 | 124% |
| Example 13 | Mixture 13 | 3.6 | 138% |
| Example 14 | Mixture 14 | 3.5 | 134% |
| Example 15 | Mixture 15 | 3.6 | 127% |
| Example 16 | Mixture 16 | 3.5 | 136% |
| Example 17 | Mixture 17 | 3.6 | 138% |
| Comparative | Comparative | 3.7 | 95% |
| Example 1 | Example D1 | | |
| Comparative | Comparative | 3.7 | 100% |
| Example 2 | Example D2 | | |

Similar results were obtained by replacing dopant K1 with the following dopants K2 and K3, and high luminescence efficiency can be obtained by using the organic mixture as described herein as a host.

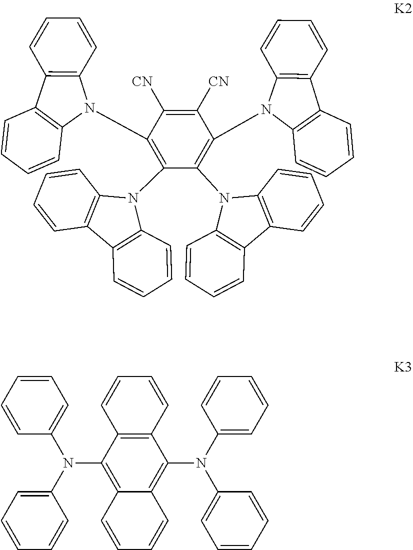

The technical features of the above-described embodiments can be combined in any ways. For the sake of brevity, not all possible combinations of the technical features of the above-described embodiments have been described. However, as long as there are no contradictions in the combination of these technical features, they should be considered to be within the scope of this specification.

What described above are several embodiments of the present disclosure, and they are specific and in details, but not intended to limit the scope of the present disclosure. It will be understood that improvements can be made without departing from the concept of the present disclosure, and all these modifications and improvements are within the scope of the present disclosure. The scope of the present disclosure shall be subject to the appended claims.

What is claimed is:

1. An organic mixture, comprising a first organic compound H₁ and a second organic compound H₂, wherein,
ΔE_{ST}(H₁)≥0.6 eV, E_{X}-T₁(H₁)≥0.6 eV; and/or
ΔE_{ST}(H₂)≥0.6 eV, E_{X}-T₁(H₂)≥0.6 eV; and
LUMO(H₂)≤LUMO(H₁)+0.10 eV;
wherein, ΔE_{ST} equals E_{S1}-E_{T1}, E_{X} equals min (|HOMO(H₁)-LUMO(H₂)|, |HOMO(H₂)-LUMO(H₁)|), E_{S1} stands for the singlet energy level, E_{T1} stands for the triplet energy level, HOMO stands for the highest occupied molecular orbital energy level, LUMO stands for the lowest unoccupied molecular orbital energy level.

2. The organic mixture of claim 1, wherein |E_{X}-E_{S1}(H₁)|≤0.4 eV, or |E_{X}-E_{S1}(H₂)|≤0.4 eV.

3. The organic mixture of claim 1, wherein both the first organic compound H₁ and the second organic compound H₂ are independently selected from the group consisting of a substituted/unsubstituted aromatic or heteroaromatic group containing 5 to 40 ring atoms, an aryloxy or heteroaryloxy group containing 5 to 40 ring atoms, and any combination thereof, wherein one or more H atoms of the foregoing groups can be further substituted with D atoms.

4. The organic mixture of claim 1, wherein the first organic compound H₁ or the second organic compound H₂ is selected from the following structures: 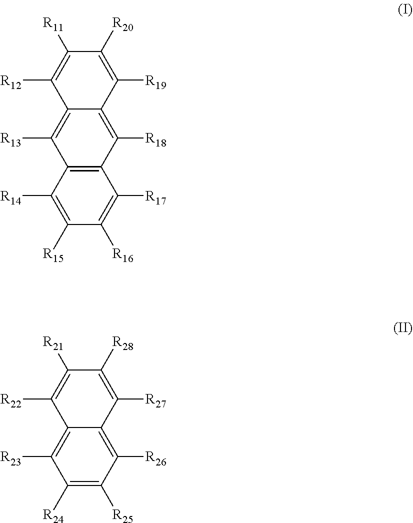
wherein R₁₁ to R₂₈ are independently selected from the group consisting of —H, —D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ substituted/unsubstituted branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic or heteroaromatic group containing 5 to 40 ring atoms, an aryloxy or heteroaryloxy group containing 5 to 40 ring atoms, and any combination thereof, wherein one or more R₁₁-R₂₈ can form a monocyclic or polycyclic aliphatic or aromatic ring system with each other and/or with the rings bonded to the R₁₁-R₂₈.

5. The organic mixture of claim 4, wherein R₁₁ to R₂₈ are independently selected from the following structures: 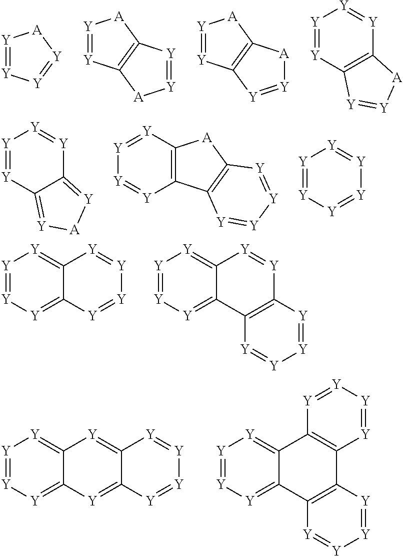
wherein,
each Y is CR₇₀₁ or N;
each A is selected from O, S, CR₇₀₂R₇₀₃, or NR₇₀₄,
R₇₀₁ to R₇₀₄ in multiple occurrences may be sites linked to other groups, or be independently selected from the group consisting of —H, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ substituted/unsubstituted branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, a CF₃ group, Cl, Br, F, a cross-linkable group, a substituted/unsubstituted aromatic or heteroaromatic group containing 5 to 40 ring atoms, an aryloxy or heteroaryloxy group containing 5 to 40 ring atoms, and any combination thereof, wherein one or more R₇₀₁-R₇₀₄ can form a monocyclic or polycyclic aliphatic or aromatic ring system with each other and/or with the rings bonded to the R₇₀₁-R₇₀₄.

6. The organic mixture of claim 1, wherein the second organic compound H₂ comprises an electron-accepting group.

7. The organic mixture of claim 2, wherein the second organic compound H₂ comprises an electron-accepting group.

8. The organic mixture of claim 3, wherein the second organic compound H₂ comprises an electron-accepting group.

9. The organic mixture of claim 4, wherein the second organic compound H₂ comprises an electron-accepting group.

10. The organic mixture of claim 5, wherein the second organic compound H₂ comprises an electron-accepting group.

11. The organic mixture of claim 1, wherein the organic mixture further comprises at least one other organic functional material, the at least one other organic functional material can be selected from the group consisting of a hole-injection material, a hole-transport material, a hole-blocking material, an electron-injection material, an electron-transport material, an electron-blocking material, an organic host material, a singlet emitting material, a triplet emitting material, a thermally activated delayed fluorescence material, and an organic dye.

12. A formulation, comprising at least one organic mixture according to claim 1 and at least one organic solvent.

13. An organic electronic device, comprising at least one organic mixture according to claim 1.

14. The organic electronic device of claim 13, wherein the organic electronic device is selected from an organic light emitting diode, an organic photovoltaic cell, an organic light emitting electrochemical cell, an organic field effect transistor, an organic light emitting field effect transistor, an organic laser, an organic spintronic electronic device, an organic sensor, or an organic plasmon emitting diode.

15. The organic electronic device of claim 13, wherein the organic electronic device is an organic electroluminescent device comprising a light-emitting layer, and the light-emitting layer comprises the at least one organic mixture.
